(19) United States
(12) Patent Application Publication
(10) Pub. No.: US 2003/0103901 A1
(43) Pub. Date: Jun. 5, 2003

(54) ELISA kit for the determination of CYP2C19 metabolic phenotypes and uses thereof

(75) Inventor: Brian Leyland-Jones, Miami, FL (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: McGill University

(21) Appl. No.: 10/131,791

(22) Filed: Apr. 23, 2002

Related U.S. Application Data
(60) Provisional application No. 60/285,245, filed on Apr. 23, 2001.

(51) Int. Cl. G01N 33/53; A61K 49/00; G01N 33/537; G01N 33/543

(52) U.S. Cl. 424/9.2; 435/7.92

(57) ABSTRACT

The invention relates to an enzyme linked immunosorbent assay (ELISA) method and kit for the rapid determination of metabolic phenotypes for Cytochrome P450 2C19 (CYP 2C19). The kit uses may include but are not limited to, use on a routine basis in a clinical laboratory to determine a Cytochrome P450 2C19 (CYP 2C19) phenotype of an individual; to allow a physician to individualize an individual's treatment with respect to the numerous drugs metabolized by CYP 2C19 based on a phenotypic determination; to predict an individual's susceptibility to carcinogen induced diseases including many cancers, and to screen individuals for a preferred metabolic phenotype in order to determine those individuals with a responsive phenotype for participation in clinical testing.

### RELATED APPLICATION

This Application claims the benefit of pending provisional application U.S. 60/285,245 filed Apr. 23, 2001, the entire teachings of which are incorporated herein by reference.

### BACKGROUND OF THE INVENTION

The invention relates to an enzyme linked immunosorbent assay (ELISA) method and kit for the rapid determination of metabolic phenotypes for Cytochrome P450 2C19 (CYP2C19). The kit uses may include but are not limited to, use on a routine basis in a clinical laboratory to determine a CYP2C19-specific phenotype of an individual; to allow a physician to individualize a treatment with respect to the numerous drugs metabolized by CYP2C19 based on a phenotypic characterization of the individual; to predict an individual's susceptibility to carcinogen induced diseases including many cancers, and to screen individuals for a preferred metabolic phenotype in order to determine those individuals with a responsive phenotype for participation in clinical testing and/or for treatment with a particular drug or class of drug compounds.

For the majority of drugs (or xenobiotics) administered to humans, their fate is to be metabolized in the liver, into a form less toxic and lipophilic with their subsequent excretion in the urine. Their metabolism involves two systems which act consecutively: the cytochrome P450 system which includes at least 20 enzymes catalyzing oxidation reactions and localized in the microsomal fraction, and the conjugation system which involves at least 5 enzymes. An enzyme of one system can act on several drugs and drug metabolites. The rate of metabolism of a drug differs between individuals and between ethnic groups, owing to the existence of enzymatic polymorphism within each system. As a result, a variety of phenotypes can be distinguished, including poor metabolizers (PM), extensive metabolizers (EM), and ultra-extensive metabolizers (UEM).

As described in U.S. Pat. No. 5,830,672, Applicant's have previously been successful in establishing an ELISA based system and method for the rapid determination of N-aceyltransferase (NAT2) phenotypes. However, to date a convenient and effective system for determining CYP2C19 phenotypes has not been provided.

In previous studies, CYP2C19 phenotypes have been generally determined by determining the ratio of the different chiral forms of mephenytoin (S & R). In these studies, the subjects ingested a racemic (50:50) dose of mephenytoin, and then the urinary concentrations of the two chiral compounds were determined by a stereo-selective, capillary gas chromatographic-based procedure (Wedlund P. J. et al. (1984) Clin Pharmacol Ther 36:773-80). Such determination methods are time-consuming, onerous, and employ systems and equipment which are not readily available in a clinical laboratory.

It would be highly desirable to be provided with a convenient and effective method for characterizing an individual's CYP2C19 phenotype using a non-toxic drug (probe substrate) so as to predict his/her response and side effects profile to a wide range of potentially toxic drugs.

It would be highly desirable to be provided with an enzyme linked immunosorbent assay (ELISA) kit for CYP2C19 phenotyping, which could be accomplished on a routine basis by any technician with a minimum of training and does not involve complex equipment.

It would also be highly desirable to be provided with an enzyme linked immunosorbent assay (ELISA) kit, which would enable a physician to individualize therapy and/or treatment. Such therapies may include treatment with drugs such as Omeprazole, Imipramine, or Pantoprazole based on an individual's CYP2C19-specific phenotype.

### SUMMARY OF THE INVENTION

One aim of the present invention is to provide an enzyme linked immunosorbent assay (ELISA) kit for the rapid determination of metabolic enzyme phenotype, which can be used on a routine basis in a clinical laboratory.

Another aim of the present invention is to provide an ELISA kit which would allow a physician to:

a) determine the CYP2C19 metabolic phenotype of an individual;

b) individualize therapies or treatments with drugs known to be dependant on CYP2C19 metabolism, according to an individual's metabolic phenotype;

c) predict an individual's susceptibility to carcinogen induced diseases such as various cancers; and

d) screen individuals for a preferred CYP2C19 metabolic phenotype in order to determine those individuals with a responsive phenotype for participation in clinical testing.

Another aim of the present invention is to provide a method of characterizing a CYP2C19-specific metabolic phenotype, wherein a plurality of phenotypic determinants are identified as corresponding to respective metabolic characteristics, said method comprising: a) administering to an individual a probe substrate specific to the CYP2C19 metabolic pathway; b) detecting metabolites of said CYP2C19 metabolic pathway in a biological sample from said individual in response to said probe substrate; and c) characterizing a phenotypic determinant of said CYP2C19 metabolic phenotype based on detected metabolites.

According to one aspect of the present invention there is provided a method of using a CYP2C19 metabolic phenotype to select a drug treatment regimen for an individual, said method comprising, comparing a metabolic profile of a candidate drug with said CYP2C19 metabolic phenotype of said individual, and selecting said candidate drug for use in said treatment regimen for said individual when said CYP2C19 metabolic phenotypic is indicative of a phenotype having metabolic efficiency for said candidate drug.

According to another aspect of the present invention there is provided a method of using a CYP2C19 metabolic phenotype to individualize a selected drug treatment regimen for an individual, wherein said CYP2C19 metabolic phenotype of said individual is determined; a safe and therapeutically effective dose of said drug treatment is determined for said individual based on said CYP2C19 phenotype; and said dose for use in said selected treatment regimen for said individual is selected based thereon.

According to another aspect of the present invention there is provided a method of treating an individual having a medical condition with a safe and therapeutically effective dose of a drug treatment known for use with said condition, said method comprising: a) determining a CYP2C19 metabolic phenotype of said individual; and b) administering a safe and therapeutically effective dose of at least one compound known for treating said condition, wherein said at least one compound known for treating said condition has a metabolic profile corresponding to said individual's metabolic phenotype for said at least one compound as represented by said CYP2C19 metabolic phenotype.

According to another aspect of the present invention there is provided a method of selecting a treatment for an individual corresponding to said individual's CYP2C19 metabolic phenotype, said method comprising: a) characterizing a CYP2C19 metabolic phenotype of said individual; b) identifying a treatment from a group of candidate treatments that corresponds to said individual's CYP2C19 metabolic phenotype; and c) selecting said treatment.

According to another aspect of the present invention there is provided a method of screening a plurality of individuals for participation in a drug treatment trial assessing the therapeutic effect of a candidate drug treatment, said method comprising: a) characterizing a CYP2C19 metabolic phenotype of each of said plurality of individuals; b) identifying those individuals having a CYP2C19 metabolic phenotype characterized as effective for metabolizing said candidate drug treatment.

According to another aspect of the present invention there is provided an assay system for detecting the presence of CYP2C19-specific metabolites in a biological sample obtained from an individual treated with a probe substrate specific for CYP2C19 metabolic pathway of said metabolites; said system comprising: a) means for receiving said biological sample, including an affinity complexation agents contained therein; b) means for detecting presence of said metabolites bound to said affinity complexation agents; and c) means for quantifying ratios of said metabolites to provide corresponding phenotypic determinants, wherein said phenotypic determinants provide a CYP2C19 metabolic phenotype profile of said individual.

According to another aspect of the present invention there is provided a method of using a CYP2C19 metabolic phenotype for determining a combination drug therapy wherein an individual's phenotype is indicative of a fast metabolizer, and a corresponding inhibitor is selected for combined treatment with a drug to improve the therapeutic effect thereof in said individual.

According to another aspect of the present invention there is provided a method of diagnosing a disease or condition associated with altered function in a drug metabolizing enzyme(s) by determining an individual's CYP2C19 metabolic phenotype.

According to another aspect of the present invention there is provided a method of determining an individual's susceptibility to a carcinogen induced disease by determining an individual's CYP2C19 metabolic phenotype.

According to another aspect of the present invention there is provided a method of determining the ability of a compound to effect the function of the CYP2C19 metabolizing enzyme(s) in a biological organism in vivo, said method comprising: a) determining a first CYP2C19 metabolic phenotype of said biological organism according to the methods of claims 1 to 25 prior to exposure to said compound; b) exposing said biological organism to said compound; c) determining a second CYP2C19 metabolic phenotype of said biological organism according to the methods of claims 1 to 25 after exposure to said compound; and d) comparing said first and second CYP2C19 phenotypes, wherein a change in said multi-determinant phenotypes determined post-compound exposure as compared to pre-compound exposure is indicative of said drug having the ability to effect the function of said CYP2C19 drug metabolizing enzyme(s) in said biological organism.

According to yet a further aspect of the present invention there are provided derivatives of S- and R-mephenytoin and uses thereof.

### BRIEF DESCRIPTION OF THE DRAWINGS

FIG. 1 illustrates chiral forms S-mephenytoin and R-mephenytoin.

FIG. 2 illustrates S-mephenytoin derivatives for CYP2C19 phenotyping by ELISA.

FIG. 3 illustrates R-mephenytoin acid derivatives for CYP2C19 phenotyping by ELISA.

FIG. 4 illustrates the scheme of the general immunosensor design depicting the intimate integration of immunological recognition at the solid-state surface and the signal transduction;

FIG. 5 illustrates the principle of SPR technology;

FIG. 6 illustrates a QCM immunosensor device;

FIG. 7 illustrates the synthetic routes for the production of AAMU and 1X derivatives used in accordance with one embodiment of the present invention;

FIGS. 8 to 11 show other AAMU and 1X derivatives which can be used for raising antibodies in accordance with another embodiment of the present invention.

FIG. 12 illustrates the absorbance competitive antigen ELISA curves of AAMU-Ab and 1X-Ab in accordance with one embodiment of the present invention;

FIG. 13 is a histogram of molar ratio of AAMU/1X;

FIG. 14 illustrates a pattern of samples to be pipetted in a 96-well microtest plate.

### DETAILED DESCRIPTION OF THE INVENTION

CYP2C19

CYP2C19 accounts for about 2% of oxidative drug metabolism. CYP2C19 has been postulated as participating in ˜8% of drug metabolism.

Polymorphism

Individuals are genetically polymorphic with respect to CYP2C19 metabolism. Two metabolic phenotypes can be distinguished: extensive and poor metabolizers. Two genetic polymorphism have been identified (CYP2C19*2 and CYP2C19*3) that together account for 100% of Oriental poor metabolizers and about 83% of Caucasian poor metabolizers. Both of these mutations introduce stop codons resulting in a truncated and non-functional enzyme.

CYP2C19 plays a role in the metabolism of a variety of compounds including tricyclic antidepressants amitriptyline, imipramine and clomipramine, the sedatives diazepam and hexobarbital, the gastric proton pump inhibitors, omeprazole, pantoprazole, and lansoprazole, as well as the antimalarial drug proguanil and the β-blocker propanolol, as exemplified in Table 1.

TABLE 1
DRUGS METABOLIZED BY CYP2C19

| Drug Class | Drugs |
|---|---|
| Anticoagulants | Warfarin |
| Antibiotics | Rifamycins |
| Antidepressants | TCA: amitryline, clomipramine, imipramine |
| | Tetracyclics: mianserin |
| | SSRI: citalopram |
| Antiepileptics | Phenobarbitol, phenytoin, S-mephenytoin, |
| | vaiproic acid |
| Antimalarials | Chiorproguanil, quinine, proguanil, |
| Antineoplastics | Alkylating: cyclophosphamide, ifosamide |
| Antiviral | AZT |
| Anxyiolitics, | Hexobarbitol, mephobarbitol |
| sedatives, hypnotics | Benzodiazepines: Diazepam, nordiazepam, |
| | temazepam |
| Gastrointestinal | Proton Pump Inhibitors: lansoprazole, |
| | omeprazole |
| Neuroleptic | Clozapine |

Induction and Inhibition

CYP2C19 is inhibited by fluconazole, fluvoxamine, fluoxetine, sertraline, ritonavir and is induced by rifampin. The ability to quickly and easily determine an individual's CYP 2C19-specific phenotype allows a physician to determine the phenotypic status of an individual and make a corresponding determination about the type and extent of treatment most suitable at a given time. The present invention provides a reliable method of identifying a suitable drug compatible with an individual's phenotype, as well as a method of individualizing therapy with a specific drug(s) with respect to dosage, duration etc. based thereon.

In accordance with an embodiment of the present invention there is provided a phenotypic determinant specific for CYP 2C19 metabolism. This phenotypic determinant provides an indication of an individual's CYP 2C19 phenotype. Furthermore, the phenotypic determinant may be used to provide a drug response profile for the individual specific to drug(s) known to be metabolized by the CYP 2C19 pathway.

Inter Ethnic Differences

The occurrence of the poor metabolizer phenotype for CYP2C19 shows a large inter ethnic variability. Poor metabolizers make up less than 4% of the European and white American populations. While the Korean population has a poor metabolizer frequency of 12.6%, the Chinese 17.4% and the Japanese 22.5%. In addition, the CYP2C19 mutant alleles demonstrate interethnic variability with CYP2C19*2 frequency ranging from 28.9% in the Chinese population to only 13% in European-American population. The CYP2C19*3 allele is absent from the European-American or African-American populations, while occurring at a frequency of 11.7% in both the Korean and Japanese populations.

It is reasonable that, in drug metabolism studies, each ethnic group can be studied separately for evidence of polymorphism and its antimode should not be extrapolated from one ethnic population to another.

Omeprazole

As an example, the benefit of CYP2C19 metabolic phenotyping in drug dosing is evident in the case of omeprazole. Omeprazole is a drug used in the treatment of H. pylori infections in conjunction with amoxicillin, and is cleared from the body via a CYP2C19 metabolic pathway. Studies have observed higher eradication rates of H. pylori in CYP2C19 poor metabolizers. Therefore, extensive metabolizers may require higher doses of omeprazole to achieve the same level of H. pylori eradication observed in poor metabolizers. For these reasons, the utility of a reliable phenotyping test for CYP2C19 is evident. In particular, an accurate and convenient clinical assay would allow physicians to quickly identify safe and effective treatment regimes for patients on an individual basis. In addition, the present invention provides a means to determine the efficiency of an individual's CYP 2C19 metabolism before prescribing a standard treatment. For example, in the event that an individual is diagnosed with an H. pylori infection, and is also being treated with fluoxetine, a physician can characterize the current state on an individual's CYP 2C19 phenotype to determine if their CYP 2C19 activity is inhibited to the extent that simultaneously treatment with H. pylori would be ineffective or potentially harmful.

Direct Phenotypic Determinants of CYP2C19

Different substrates (or probe drugs) can be used to determine a CYP 2C19 phenotype according to the present invention. Particularly suitable probe drugs include without limitation, mephenytoin, omeprazole or chloroguanil. Of these, mephenytoin is the preferred probe drug.

In accordance with an embodiment of the present invention, the chiral ratio of S-mephenytoin and R-mephenytoin in a urine sample may be used to provide a determination of an individual's CYP2C19 phenotype. These forms of mephenytoin are used as quantitative markers in the determination of a CYP2C19 phenotype on the basis of the use of the preferred probe drug mephenytoin. However, it is fully contemplated that the present invention is not limited in any respect thereto.

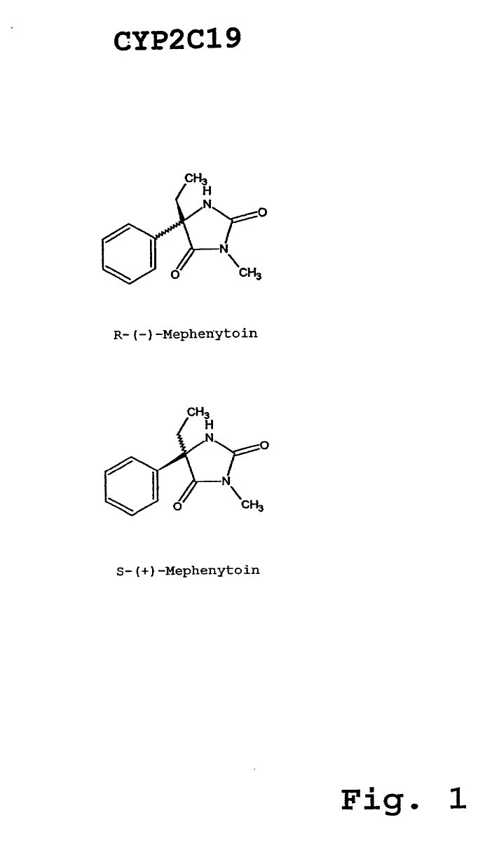

The structure of R−(−) and S−(+) mephenytoin and are illustrated in FIG. 1.

The chiral ratio of S-mephenytoin and R-mephenytoin, used to determine the CYP2C19 phenotype of the individual, is as follows:

$$\frac{S \text{-} Mephenytoin}{R \text{-} Mephenytoin}$$

Chiral ratios of close to unity (>0.8) are indicative of fast CYP2C19 metabolizers.

Enzyme linked immunosorbent assays (ELISA) have been successfully applied in the determination of low amounts of drugs and other antigenic compounds in plasma and urine samples and are simple to carry out. An ELISA for N-acetyltransferase-2 (NAT2) phenotyping using caffeine as a probe drug has also been developed and validated (Wong, P., Leyland-Jones, B., and Wainer, I. W. (1995) J. Pharm. Biomed. Anal. 13: 1079-1086); (Leyland-Jones et al. (1999) Amer. Assoc. Cancer Res. 40: Abstract 356). The ELISA for NAT2 phenotyping is simpler to carry out than the HPLC and CE.

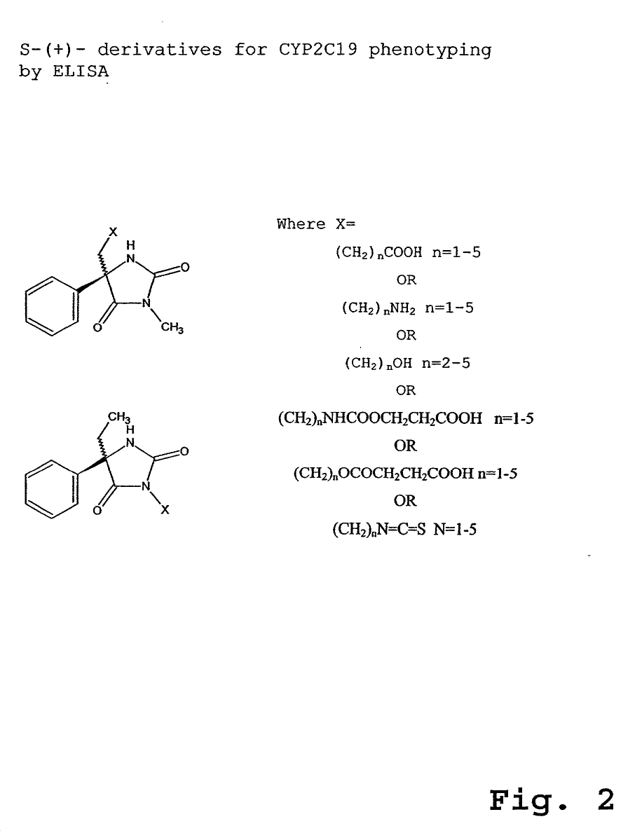

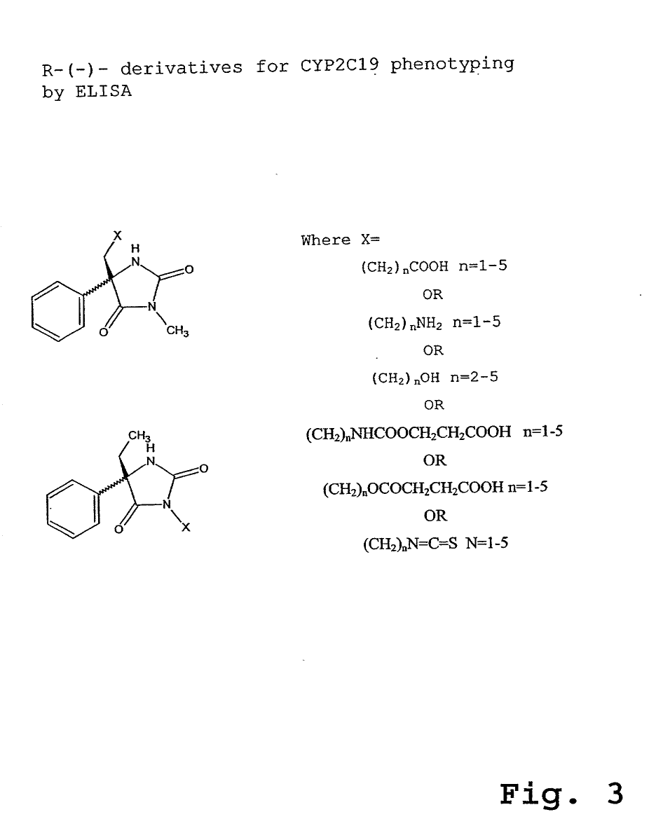

Antibodies to S-mephenytoin and R-mephenytoin have been developed in order to measure the chiral ratio of these forms of mephenytoin in urine samples collected from an individual after racemic (50:50) mephenytoin consumption. In addition, an antigen enzyme linked immunosorbent assay (ELISA) for measuring the ratio of S-mephenytoin and R-mephenytoin in a urine sample using these antibodies is provided. The antibodies of the present invention can be polyclonal or monoclonal antibodies raised against derivatives of S-mephenytoin and R-mephenytoin, as exemplified in FIGS. 2 and 3, which allow the measurement of the chiral ratio (S:R) of mephenytoin.

In accordance with an embodiment of the present invention, the ratio of S-mephenytoin and R-mephenytoin in a urine sample may be used to provide a determination of an individual's CYP 2C19 phenotype. These forms of mephenytoin are used as quantitative markers in the determination of a CYP 2C19 phenotype on the basis of the use of the preferred probe drug mephenytoin. However, it is fully contemplated that the present invention is not limited in any respect thereto.

In accordance with another embodiment of the present invention, a competitive antigen ELISA is provided for determining CYP 2C19 phenotyping using mephenytoin as the probe drug. The assay is sensitive, rapid and can be readily carried out on a routine basis by a technician with a minimum of training in a clinical laboratory.

Ligand-Binding Assays

The specificity of the molecular recognition of antigens by antibodies to form a stable complex is the basis of both the analytical immunoassay in solution and the immunosensor on solid-state interfaces. The underlying fundamental concept of these analytical methods as ligand-binding assays is based on the observation of the products of the ligand-binding reaction between the target analyte and a highly specific binding reagent.

The development of immunoassay technology is a success story especially for the clinical laboratory and still continues to be a vibrant area of research. Further development and automation will expand the possibilities of immunoassay analysis in the clinical sciences. Besides this, new areas for trace analyses using immunoassay were defined in the last decade: the environmental analysis of trace substances and quality control in the food industry. Since these applications also need a continuous monitoring mode, the idea of an immunosensor as a continuously working heterogeneous immunoassay system, covering these features, was conceived. The immunosensor is now considered as a major development in the immunochemical field. Despite an overwhelming number of papers is this field, there are only a few commercial applications of immunosensors in clinical diagnostics. The reasons are, in part, unresolved fundamental questions relating to immobilization, orientation, and specific properties of the antibodies or antibody-related reagents on the transducer surface. In addition, a key issue is which clinical applications may benefit most from immunosensor devices in the routine medical laboratory. Only if there is consensus on the clinical utility of this new technique can the gap between the high expectations of the developer and reality be closed. Designers of immunosensor devices must be aware of the general and special needs of laboratory medicine from new analytical techniques.

A new analyzer should be simple and “rugged” for the measurement of analytes. Measurements have to be performed precisely and accurately, even under emergency conditions. The analyzer must be fully automated and capable of performing rapid measurements with turnaround times of <1 h. Additionally, the determination of an analyte should preferably be without sample pretreatment in matrices, such as serum, plasma, urine or cerebrospinal fluid. All parameters determined with a new analyzer must meet the following criteria, which are defined in various guidelines: low imprecision, small lot-to-lot variations, high analytical sensitivity, optimum analytical specificity and accuracy with long calibration stability and low interferences by drugs or normal and pathological sample components.

In the clinical laboratory, a future substitution of immunoassays by immunosensors simply depends on the superiority and versatility of the new methodology. The applicability for point-of-care testing or when they are temporarily implanted into the individual additionally depends on the reliable and accurate analysis of the desired analyte, without drift problems or matrix interferences. Due to the tremendously growing variety of developments, this review is not intended to be comprehensive. Hence, the main focus will be the description and assessment of reported clinical applications of immunosensors. For a more thorough understanding, we refer to several excellent reviews in the last 5 years on technical aspects and the application of immunosensors in various fields. Other related reviews deal with antibody engineering developments and latest immunoassay technologies.

Antibodies as Bioaffinity Interface for Both Immunoassays and Immumosensors

It should first be clarified that the specificity for the measurement of analytes in all immunosensor systems, as in the case of immunoassays, is dependent on the application of affinity complexation agents (binding molecules). This pivotal feature is shared by both technologies. New developments in protein engineering for immunoglobulins (including antibody fragments, and chimeric antibodies) or in substituting antibodies by alternative binding components (aptamers are one example) or structures (molecular imprinting is one example) will, therefore, be applicable to either technology, if available. In particular, the possibilities in antibody engineering will enable changes in the affinity and fine specificity of antibodies, as well as the expression of fragments as fusion proteins coupled to reporter molecules.

Immobilization Procedures for Antibodies

Antibodies have to be properly immobilized on the immunosensor surface, which is mostly part of a flow-through cell. The optimum density and adjusted (but not random) orientation of the antibodies are of paramount importance. Due to the different types of sensing surfaces, this manipulation can have benefits e.g., improvement of the reaction kinetic parameters, but also unfavorable effects (e.g., increased nonspecific binding, partly destroyed paratope). There are four different types of oriented coupling of antibodies: binding to Fc receptors such as protein A or G or recombinant ArG fusion protein on the surface; binding of other binding partners to structures, covalently linked to the Fc part of the antibody, e.g., the biotin residue on the Fc binds to surface-coated streptavidin; coupling to the solid support via an oxidized carbohydrate moiety on the C2 Fc domain; and the binding of Fab or scFv fragments to the surface of the device via a sulfhydryl group in its C-terminal region.

Numerous chemical reactions can be applied to the immobilization onto solid surfaces. Defined linkages between the antibody or its carbohydrate moieties and the solid phase material (silica, silanized silica, Ta- or Ti-oxides, plastics, sepharose, and metal films) are being built by glutaraldehyde, carbodiimide, uccinimide ester, maleinimide, periodate or galactose oxidase. Moreover, photo-immobilization of antibodies using albumin derivatized with aryldiaziridines as photolinker, is applicable. Physiosorption is not recommended due to the local instability of the layer caused by the mechanical stress in the flow-through cell. An exciting new method for antibody immobilization on a quartz surface of a piezoelectric sensor is based on the deposition of an ethylenediamine plasma polymerization film on the quartz crystal. This film is extremely thin and homogeneous, incorporating amino functions which may be further derivatized and linked to immunoglobulins, resulting in an orientation-controlled and highly reusable sensing surface. Another recent development is the planar-supported phospho-lipid bilayer (SLB), which can be formed on solid supports by vesicle fusion and Langmuir-Blodgett methods. SLBs maintain two-dimensional fluidity and accommodate multivalent binding between surface-bound ligands and receptor molecules in solution.

For noble metal surfaces, such as gold, in particular, in optical immunosensors, self-assembling monolayer (SAM) techniques seem to be first choice. In general, a SAM is built of long-chained (C₁₂ and higher) n-alkylthiols with derivatized organic functional groups, which are easily linked to the gold film via the thiol groups by a mechanism still not fully understood. The functional groups of the SAM cross-link with the Fc portion of the antibody (one way is via the biotin streptavidin system), whereas the self-organization of the matrix prevents the surface being individualed?? to nonspecific binding effects. In addition, the covalent coupling of IgG to a short-chain (thioctic or mercaptopropionic acid are two examples) SAM-modified metal surface has been shown to be an effective affinity-based layer for optical immunosensors.

Regeneration of Antibody-coated Sensor Surfaces

Conventional homogeneous and heterogeneous immunoassays, respectively, work discontinuously. It is highly desirable, however, that immunosensor devices, applied in clinical diagnostics, are capable of quasi-continuous recording. The repeated use of disposable sensing elements may mimic a pseudocontinuous action, but this is not considered here. In true immunosensors, the analyte/antigen interaction on the sensor-coated surface is reversible. With the given short incubation times in the flow-through device, the reaction between antigen and antibody is far off the equilibrium state. Fast reversibility and high sensitivity are mutually exclusive of each other. Consistently, an adequate analytical sensitivity is only warranted if antibodies with increased affinity >10¹⁰ M⁻¹ or at least with highly improved on-rate are applied.

The regeneration of the binding sites of the antibodies bound to the immunosensor surface needs stringent procedures. Antibody regeneration using acidic or alkaline solutions, guanidinium chloride, or ionic strength shock is potentially harmful to the binding ability and may lead to a diminished lifetime of the immobilized antibodies and insidious drift problems.

Besides this, it must be considered that with the short reaction times between the antibodies and soluble analytes in the flow-through system, the cross-reactivities of the antibody applied can be increased. A highly specific recognition of the antigen is a kinetic-controlled process due to the complexity of the conformational changes in the Fab portion of the antibody upon binding of the antigen.

There are different approaches to solve the “antibody regeneration” problem: one approach is to displace the antigenic analyte by a highly concentrated solution of a related antigen with weak affinity to the surface-bound antibody. However, this depends on the availability of a suitable antigenic surrogate. This is not always feasible and is only applicable to small analytes. A second approach is to use the techniques of antibody engineering to improve the chemical stability of antibodies as whole molecules or as Fab fragments. The phage display technique is such a powerful tool. This can be helpful in the selection of antibody fragments with improved stability. Libraries of mutants of single-chain Fv fragments (scFv), comprising the variable regions of the L and H chains, joined by a peptide linker are generated by a combination of site-directed and random mutagenesis. The selection can be carried out under different physical or chemical pressures to produce thermodynamically more stable scFv mutants. An interesting third approach is a pseudo-regenerating procedure for immunosensors. An amperometric sensor is coated with a conducting immunocomposite, formed by a mixture of specific antibody with methacrylate monomer and graphite. After polymerization, the device is ready for use. Repeated measurements became possible if the polymer is polished thoroughly with abrasive paper. These notes do not apply to immunosensors with a competitive configuration, in which antigenic compounds and not antibodies are surface-immobilized.

Alternative Analyte-binding Compounds for Immunosensor Applications

Aptamers

Aptamers are single-stranded DNA or RNA oligonucleotide sequences with the capacity to recognize various target molecules with high affinity and specificity. These ligand-binding oligonucleotides mimic properties of antibodies in a variety of diagnostic formats. They are folded into unique overall shapes to form intricate binding furrows for the target structure. Aptamers are identified by an in vitro selection process known as systematic evolution of ligands by exponential enrichment (SELEX). Aptamers may have advantages over antibodies in the ease of depositing them on sensing surfaces. Moreover, due to the highly reproducible synthetic approach in any quantities, albeit the affinity constants are consistently lower than those of antibodies and the stability of these compounds is still questionable, they may be particularly useful for diagnostic applications in complex biological matrices. The aptamer-based schemes are still in their infancy and it is expected that modified nuclease-resistant RNA and DNA aptamers will soon be available for a variety of therapeutic and diagnostic formats. The potential of aptamers for use in biosensors has been outlined in the design of a fiber-optic biosensor using an anti-thrombin DNA aptamer, immobilized on the surface of silica microspheres and distributed into microwells on the distal tip of the imaging fiber. With this device, the determination of thrombin at low concentration was possible. Exciting new possibilities are evolving by the introduction of signaling aptamers with ligand-dependent changes in signaling characteristics and catalytically active so-called “apta-zymes” which would allow the direct transduction of molecular recognition to catalysis.

Anticalins

Lipocalins constitute a family of proteins for storage or transport of hydrophobic and/or chemically sensitive organic compounds. The retinol-binding protein is an example in human physiology. It has been demonstrated that the bilin-binding protein, a member of the lipocalin family and originating from the butterfly Pieris brassicae, can be structurally reshaped in order to specifically complex potential antigens, such as digoxigenin, which was given as an example. These binding proteins share a conserved β-barrel, which is made of eight antiparallel β-strands, winding around a central core. At the wider end of the conical structure, these strands are connected in a pairwise manner by four loops that form the ligand binding site. The lipocalin scaffold can be employed for the construction of so-called “anticalins”, which provide a promising alternative to recombinant antibody fragments. This is made by individualizing various amino acid residues, distributed across the four loops, to targeted random mutagenesis. It remains to be shown that this class of proteins is applicable in diagnostic assays and in immunosensors. Critical points that still need to be defined include the synthesis and stability of the anticalins, the magnitude of the affinity constants, and the versatility for being crafted against the large variety of ligands.

Molecular Imprinting Techniques

This is a technique that is based on the preparation of polymeric sorbents which are selectivity predetermined for a particular substance, or group of structural analogs. Functional and cross-linking monomers of plastic materials, such as methacrylics and styrenes, are allowed to interact with a templating ligand to create low-energy interactions. Subsequently, polymerization is induced. During this process, the molecule of interest is entrapped within the polymer either by a noncovalent, self-assembling approach, or by a reversible, covalent approach. After stopping the polymerization, the template molecule is washed out. The resultant imprint of the template is maintained in the rigid polymer and possesses a steric (size, shape) and chemical (special arrangement of complementary functionality) memory for the template. The molecularly imprinted polymer (MIP) can bind the template (=analyte) with a specificity similar to that of the antigen-antibody interaction.

Besides the main applications in solid-phase extraction and chromatography, molecularly imprinted polymers have already been employed as nonbiological alternatives to antibodies in competitive binding assays. A series of applications for analytes, such as cyclosporin A, atrazine, cortisol, 17b-estradiol, theophylline, diazepam, morphine, and S-propranolol, suggests that molecular imprinting is a promising technique for immunoassays and immunosensors.

Immunoassay and Immunosensor Technologies

Immunoassays

Immunoassays use antibodies or antibody-related reagents for the determination of sample analytes. This analytical tool has experienced an evolutionary history since 1959, when Berson and Yalow first described the radioimmunoassay (RIA) principle. In the RIA, a fixed and limited amount of antibody is reacted with a fixed and limited amount of radiolabeled antigen tracer and a variable concentration of the analyte. The selectivity of the ligand-binding of antibodies allows these biomolecules to be employed in analytical methods that are highly specific even in complex biological matrices, such as blood, plasma, or urine. By combining the selectivity of antibody-analyte interactions with the vast array of antibodies preformed in immunization processes of host animals and the availability of numerous readily detectable labels radioisotopes, enzymatically or electrochemically induced adsorbance or fluorescence or chemi-luminescence, immunoassays can be designed for a wide variety of analytes while with extraordinarily low detection limits.

Biosensors and Immunosensors

A biosensor is an analytical device that integrates a biological element on a solid-state surface, enabling a reversible biospecific interaction with the analyte, and a signal transducer. The biological element is a layer of molecules qualified for biorecognition, such as enzymes, receptors, peptides, single-stranded DNA, or even living cells. If antibodies or antibody fragments are applied as a biological element the device is called an immunosensor. Compared to conventional analytical instruments, biosensors are characterized by an integrated structure of these two components. Many devices are connected with a flow-through cell, enabling a flow-injection analysis (FIA) mode of operation. Biosensors combine high analytical specificity with the processing power of modern electronics to achieve highly sensitive detection systems.

There are two different types of biosensors: biocatalytic and bioaffinity-based biosensors. The biocatalytic biosensor uses mainly enzymes as the biological compound, catalyzing a signaling biochemical reaction. The bioaffinity-based biosensor, designed to monitor the binding event itself, uses specific binding proteins, lectins, receptors, nucleic acids, membranes, whole cells, antibodies or antibody-related substances for biomolecular recognition. In the latter two cases, these biosensors are called immunosensors.

Biosensors are extensively used as diagnostic tools, predominately in point-of-care testing. Probably the most successful commercialization of biosensors today is the in vitro near individual measurement of capillary glucose using various hand-held systems with disposable reagent cartridges.

Immunosensor Principles

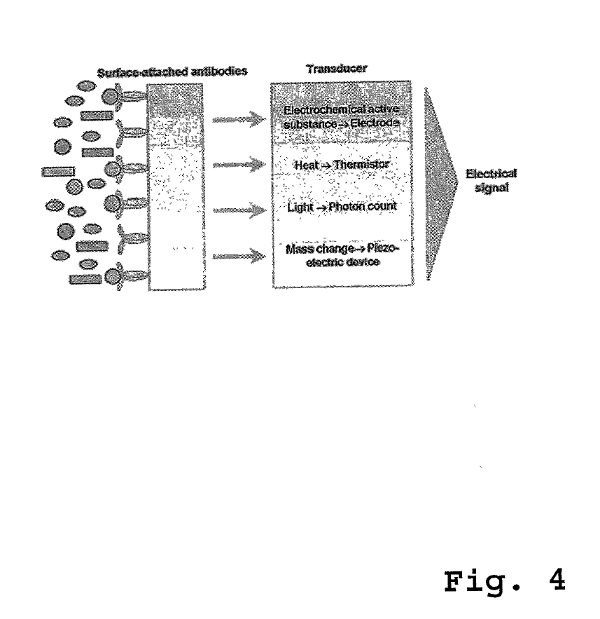

The general immunosensor design is depicted in FIG. 4. There are four types of immunosensor detection devices: electrochemical (potentiometric, amperometric or conductometric/capacitative), optical, microgravimetric, and thermometric. All types can either be run as direct nonlabeled or as indirect labeled immunosensors. The direct sensors are able to detect the physical changes during the immune complex formation, whereas the indirect sensors use signal-generating labels which allow more sensitive and versatile detection modes when incorporated into the complex.

There is a great variety of different labels which have been applied in indirect immunosensors. In principle they are the same labels as used in immunoassays. Among the most valuable labels are enzymes such as peroxidase, glucose oxidase, alkaline phosphatase (AP), catalase or luciferase, electroactive compounds such as ferrocene or In²+ salts, and a series of fluorescent labels (including rhodamine, fluorescein, Cy5, ruthenium diimine complexes, and phosphorescent porphyrin dyes). In particular, laser-induced fluorometric resonance energy transfer between two fluorophores offers methodological advantages and can be extended to fiberoptic sensing.

Although indirect immunosensors are highly sensitive due to the analytical characteristics of the label applied, the concept of a direct sensor device is still fascinating and represents a true alternative development to immunoassay systems. Its potential simplicity holds multiple advantages, making immunosensors progressive and future directed.

The present invention will be illustrated using the following examples, which are not to be seen as limiting in any way. Those skilled in the art will recognize, or be able to ascertain using no more than routine experimentation, many equivalents to the specific embodiments of the invention described specifically herein Such equivalents are intended to be encompassed in the scope of the claims.

Electrochemical Sensors

Potentiometric Immunosensors. The Nernst equation provides the fundamental principle of all potentiometric transducers. According to this equation, potential changes are logarithmically proportional to the specific ion activity. Potentiometric transducer electrodes, capable of measuring surface potential alterations at near-zero current flow, are being constructed by applying the following methodologies.

Transmembrane Potential. This transducer principle is based on the accumulation of a potential across a sensing membrane. Ion-selective electrodes (ISE) use ion-selective membranes which generate a charge separation between the sample and the sensor surface. Analogously, antigen or antibody immobilized on the membrane binds the corresponding compound from the solution at the solid-state surface and changes the transmembrane potential.

Electrode Potential. This transducer is similar to the transmembrane potential sensor. An electrode by itself, however, is the surface for the immunocomplex building, changing the electrode potential in relation to the concentration of the analyte.

Field-Effect Transistor (FET). The FET is a semiconductor device used for monitoring of charges at the surface of an electrode, which have been built up on its metal gate between the so-called source and drain electrodes. The surface potential varies with the analyte concentration. The integration of an ISE with FET is realized in the ion-selective field-effect transistor (ISFET). This technique can also be applied to immunosensors.

An advantage of potentiometric sensors is the simplicity of operation, which can be used for automation, and the small size of the solid-state FET sensors. All potentiometric methods, however, are still suffering from major problems of sensitivity, being inferior to amperometric transducers and nonspecific effects of binding or signaling influences from other ions present in the sample. Especially, the signal-to-noise ratio causes analytical problems, which are difficult to circumvent. Thus, a trend away from these techniques has been observed in the last few years. However, the ISFET may be seen as a candidate for ultrasensitive clinical immunosensor applications, in particular, when the novel concept of differential ISFET-based measurement of the zeta potential is used. The streaming potential is a potential difference in flow direction, caused by the flow of excess ions resulting from a local distortion of the charge balance. The zeta potential, directly correlated to the streaming potential, reflects the potential changes in the diffuse outer layer at the solid-liquid interface. It efficiently reacts to protein accumulations onto sensor surfaces and, thus, is suitable for detecting immunocomplex reactions.

Amperometric Immunosensors

Amperometric immunosensors are designed to measure a current flow generated by an electrochemical reaction at constant voltage. There are only few applications available for direct sensing, since most protein analytes are not intrinsically able to act as redox partners in an electrochemical reaction. Therefore, electrochemically active labels directly or as products of an enzymatic reaction are needed for the electrochemical reaction of the analyte at the sensing electrode. Oxygen and H₂O₂ electrodes are the most popular. An oxygen electrode consists of an electrolyte-bearing chamber with a sensing Pt cathode, polarized at 0.7 V, and an Ag/AgCl reference electrode. The chamber is gas-permeable, covered by an 0₂-pervious membrane.

Besides oxygen, generated by catalase from H₂O₂ there are other amperometrically detectable compounds, such as ferrocene derivatives or In salts. A novel approach is the use of the redox polymer [PVP-Os(bipyridyl)₂Cl), which is coimmobilized with specific antibodies. Additionally, there are examples for enzymes with electrochemically active products. AP, for example, catalyzes the hydrolysis of phenyl phosphate or p-aminophenyl phosphate (4-APP) compounds, which result in electrochemically active phenol or p-aminophenol. Furthermore, enzymes, such as horseradish peroxidase (HRP), glucose oxidase, glucose-6-phosphate dehydrogenase, with subsequent amperometrical oxidation of NADH and others, have also been successfully applied as labels.

The main disadvantage for amperometric immunosensors of having an indirect sensing system, however, is compensated for by an excellent sensitivity. This is due to a linear analyte concentration range compared to a logarithmic relationship in potentiometric systems. Special attention must be directed to the system-inherent transport rate limitations for redox partners on the electrode surface.

Conductometric and Capacitive Immunosensors

These immunosensor transducers measure the alteration of the electrical conductivity in a solution at constant voltage, caused by biochemical enzymatic reactions which specifically generate or consume ions. The capacitance changes are measured using an electrochemical system, in which the bioactive element is immobilized onto a pair of noble metal, mostly Au or Pt, electrodes. There are only few clinical applications available, as the high ionic strength of biological matrices makes it difficult to record the relatively small net conductivity changes caused by the signaling reaction. To circumvent this problem, recently, an ion-channel conductance immunosensor, mimicking biological sensory functions, was developed. The basis of this technique is the fact that the conductance of a population of molecular ion channels, built of tethered gramicidin A and aligned across a lipid bilayer membrane, is changed by the antibody-antigen binding event. Different applications using various antibodies, linked to the ion-channel complex, are given.

Another approach is the measurement of changes of the surface conductivity. For example, a conductometric immunosensor for the determination of methamphetamine (MA) in urine was recently developed. Anti-MA antibodies were immobilized onto the surface of a pair of platinum electrodes. The immunocomplex formation caused a decrease in the conductivity between the electrodes. The measurement of the reciprocal capacitance, performed at alternating voltage, is advantageous compared to conductometric devices, and serves two purposes. The first is to test the insulating monolayer on the sensor noble metal surface. Self-assembling monolayers, have insulating properties. Besides this, they prevent the immunosensor from being affected by nonspecific binding phenomena. Even minor desorption of the monolayer results in an essential increase in capacitance. Thus, the actual quality of the device can be checked. The second application is the measurement of changes in the effective dielectric thickness of the insulating layer during antigen binding, when antibodies are linked to the alkylthiol layer. Of course, this is on condition that the v-substitution of the alkylthiol monolayer does not compromise the insulation. Hence, a marked decrease of the electrical capacitance is observed and is used to quantitate the analyte. The destructive influence of lateral diffusion on nanostructured monolayers is prevented by use of the spreader-bar technique.

Optical Sensors

Optical immunosensors are most popular for bioanalysis and are today's largest group of transducers. This is due to the advantages of applying visible radiation compared to other transducer techniques. Additional benefits are the nondestructive operation mode and the rapid signal generation and reading. In particular, the introduction of fiber bundle optics (“optodes”) as optical waveguides and sophisticated optoelectronics offers increased versatility of these analytical devices for clinical applications.

Changes in adsorption, fluorescence, luminescence, scatter or refractive index (RI) occur when light is reflected at sensing surfaces. These informations are the physical basis for optical sensor techniques. Usually, applied detectors are photodiodes or photomultipliers.

There are numerous applications of either direct label-free optical detection of the immunological reaction, of labeled immunospecies, or of the products of enzymatic reactions. Most labels are fluorescent, but bio- and chemiluminescence species are also possible. It is worth mentioning that the label-free evanescence wave-related sensors explicitly represent an elegant methodology, which is a valuable alternative to sophisticated immunoassays. Nevertheless, label-free systems are prone to unsolved problems, such as nonspecific binding effects and poor analytical sensitivity to analytes with low molecular weight. Kubitschko et al. noted that despite the efforts, all immunosensors are still one magnitude less sensitive than commercial immunoassays for determining analytes in human serum, particularly those with low molecular weight. They claim the use of mass labels, such as latex particles, in order to enhance the signal. The authors demonstrated the optimization of a nanoparticle enhanced bidiffractive grating coupler immunosensor for the detection of thyroid-stimulating hormone (TSH, MW 28,000 Da). The excellent performance characteristics of this sensor clearly showed how future devices should work. The problem of unspecific binding, however, can also be controlled by applying a reference sensing region on the chip.

Total Internal Reflection Spectroscopy (TIRS)

The common principle of the following analytical devices is that in an optical sensor with two materials with different refractive indices (RI), total internal reflection occurs at a certain angle of the light beam being directed through the layer with the higher RI towards the sensing interface. By this, an evanescence wave is generated in the material with the lower RI. This wave, being an electrical vector of the wavelength of the incident light beam, penetrates further into the medium with exponentially attenuated amplitude. Biomolecules attached in that portion of the medium will interact inevitably with the evanescent wave and, therefore, lead to a distinctive diminution of the reflected light. The resolution is directly proportional to the length of interaction. Infrared spectroscopes, measuring attenuated total reflectance, are commonly built in the Kretschmann configuration: an optically absorbing film at the sensor's surface enables the measurement of the attenuated light intensity as a function of the wavelength of the incident beam. For total internal reflection fluorescence (TIRF), analytics benefit from the fact that incident light excites molecules with fluorescence characteristics near the sensor surface creating a fluorescent evanescent wave. The emerging fluorescence is finally detected. The technique has been developed mainly for an optical detection of fluorescence-labeled antibodies or antigens. In the latter case, the fluorescence capillary fill device (FCFD) technique is worth mentioning. The FCFD is designed by using a planar optical waveguide and a glass plate separated from each other by a capillary gap. Fluorophore-labeled antigen is attached on the surface of the glass plate, whereas antibodies are immobilized on the surface of the optical waveguide.

Another phenomenon, the optical diffraction, is used by the optical biosensor assay (OBA™) system: biomolecules are attached to the surface of a silanized wafer. The protein-coated surface is illuminated through a photo mask to create distinct periodic areas of active and inactive protein. Upon illumination with laser light, the diffraction grating caused by the ligand-binding process diffracts the incident light. An analyte-free negative sample does not result in diffraction because no antigen-antibody binding occurred creating the diffraction grating. The presence or absence of a diffraction signal differentiates between positive and negative samples. The intensity of the signal provides a quantitative measure of the analyte concentration.

Ellipsometry

If linearly polarized light of known orientation is reflected at oblique incidence from a surface, the reflected light is elliptically polarized. The shape and orientation of the ellipse depend on the angle of incidence, the direction of the polarization of the incident light, and the reflection properties of the surface. On adsorption of biomolecules onto a planar solid surface, phase and amplitude of the reflected light are altered and can be recorded by ellipsometric techniques. These changes in the polarization of the light are due to the alterations of the RI and the coating thickness. There are only few applications, such as the study of a cholera toxin-ganglioside GM1 receptor-ligand reaction, which were carried out using an ellipsometer.

Optical Dielectric Waveguides

Optical waveguides are glass, quartz or polymer films or fibers made of high RI material embedded between or in lower index dielectric materials. If a linearly polarized helium-neon laser light wave, introduced into the high index film or fiber, arrives at the boundary at an angle which is greater than the critical angle of total reflection, it is confined inside the waveguide. Similar to surface plasmon resonance, an evanescent field develops at the sensor's surface. In this case, however, the evanescent field is generated by the excitation of the light itself in the dielectric layer. Most of the laser light is transmitted into the device and multiple reflections occur as it travels through the medium if a bioactive substance is placed over the surface. Some of the light, however, penetrates the biolayer. This light is reflected back into the waveguide with a shift in phase interfering with the transmitted light. Thus, changes in properties of the biolayer can be followed by detecting the changes in interference.

Waveguides are often made in the form of fibers. These fiber-optic waveguide systems offer advantages for sensors when being used for hazardous analysis. Planar waveguide systems are also applicable for interferometers. They use laser light directed towards the surface of the waveguide with the attached biomolecules, which is subsequently split into two partial electrical (TE) and magnetic (TM) fieldwaves, perpendicular to each other. The interaction with the sample surface changes the relative phase between TE and TM by the different RI and surface thickness values. Various configurations, such as the Fabry-Perot monomode channel interferometer, the Mach-Zehnder interferometer or the related two-mode thin-film waveguide difference interferometer, have been successfully established.

Another technique uses thin corrugations etched into the surface of a waveguide. This grating coupler device allows the measurement of the coupling angle of either the input or output laser beam. Both beams are correlated to the RI within the evanescent field at the sensor's surface. Recently, a long-period grating fiber immunosensor has proven to be sensitive (enabling analyses down to the nanomolar range) and reproducible. Grating couplers are also used for optical waveguide lightmode spectroscopy (OWLS). The basic principle of the OWLS method is that linearly polarized light is coupled by a diffraction grating into the waveguide layer. The incoupling is a resonance phenomenon that occurs at a defined angle of incidence that depends on the RI of the medium covering the surface of the waveguide. In the waveguide layer, light is guided by total internal reflection to the edges where it is detected by photodiodes. By varying the angle of incidence of the light, the mode spectrum is obtained from which the effective RIs are calculated for both TE and TM.

Surface Plasmon Resonance (SPR)

Among the different detection systems, SPR is the most popular one. There are two leading systems on the market: the BIAcore™ systems from Biacore (Uppsala, Sweden) and the IAsys™ from Fisons Applied Sensor Technology (Cambridge, UK). Other systems with small market positions are the BIOS-1 from Artificial Sensing Instruments (Switzerland), the SPR-20 from Denki Kagaku Keiki (Japan), the SPEETA from Texas Instruments (USA), the IBIS from Windsor Scientific (UK) and the DPX from Quantech (USA). The first two commercial evanescence-wave devices are widespread in research laboratories due to the sophisticated apparatus and userfriendly control software. The BIAcore™, however, has the biggest market position.

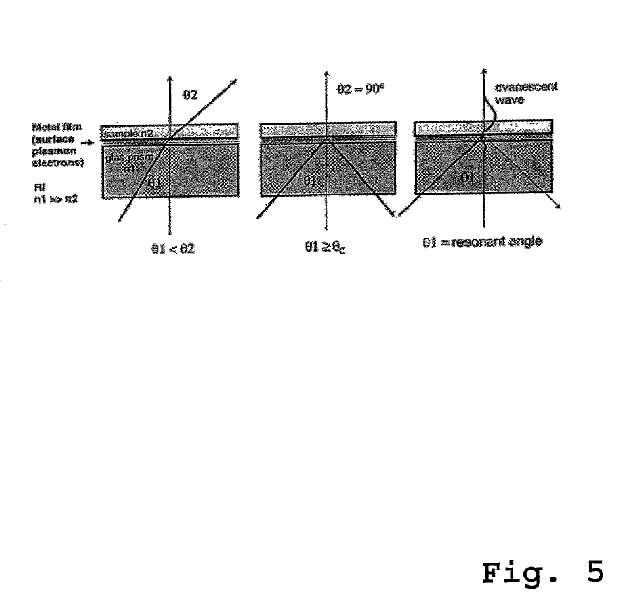

The general principle of SPR measurement 80 is depicted in FIG. 5. Polarized light is directed from a layer of high RI towards a layer with low RI to result in total internal reflection. The sample is attached to the layer of low RI. At the interface between the two different media, a thin approximately 50 nm gold film is interposed. Although light does not propagate into the low RI medium, the interfacial intensity is not equal to zero. The physical requirement of continuity across the interface is the reason for exciting the surface electrons “plasmons” in the metal film by the light energy. As a result, the electrons start oscillating. This produces an exponentially decaying evanescent wave penetrating a defined distance into the low RI medium, which is accountable for a characteristic decrease in the intensity of the reflected light. Hence, a direct insight in changes of the RI at the surface interface is made possible by monitoring the intensity and the resonance angle of the reflected light, caused by the biospecific interactions which took place there. Whereas in the BIAcore™ system, the light affects the sensing layer only once, there are several propagation contacts in the IAsys™ due to the device's resonant mirror configuration. The BIAcore™ SPR apparatus is characterized by a sensitive measurement of changes of the RI when polarized laser light is reflected at the carboxy-methylated dextranactivated device interface. The IAsys™ SPR device also uses a carboxy-methylated dextran-activated surface. Its dextran layer, however, is not attached to a gold surface, but to titanium, which forms a high refractive dielectric resonant layer. The glass prism is not attached tightly on the opposite side of the titanium layer, making space for an interposed silica layer of low RI. By this layer, the laser light beam couples into the resonant layer via the evanescent field. Therefore, the IAsys™ is seen as a combination of SPR resonant mirror with waveguide technology. As a result, no decrease in the reflected light intensity at resonance is observed in this system. The specific signal is the change in the phase of the reflected polarized light.

Differential SPR, a novel modification of a SPR immunosensor, improves further the sensitivity of the sensor by applying a modulation of the angle of light incidence. The reflectance curve is measured with a lock-in amplifier and recorded in the first and second derivative.

Light is directed from a prism with a RI towards a layer with low RI, resulting in total internal reflection. Although light does not propagate into the medium, the interfacial intensity is not equal to zero. Physical requirements of continuity across the interface are the cause of excitation of surface plasmons in the metal film by the light energy, causing them to oscillate. This produces an exponential evanescent decaying, which penetrates a defined distance into the low-index medium and results in a characteristic decrease in reflected light intensity.

Microgravimetric Sensors

A direct measurement of mass changes induced by the forming of antigen/antibody complexes is also enabled by acoustic sensors. The principle of operation is based on the propagation of acoustic shear waves in the substrate of the sensor. Phase and velocity of the acoustic wave are influenced by the specific adsorption of antibody molecules onto the antigen-coated sensor surface. Piezoelectric materials, such as quartz (SiO₂), zinc oxide (ZnO) or others resonate mechanically at a specific ultrasonic frequency in the order of tens of megahertz when excited in an oscillating electrical field. The resonant frequency is determined by the distance between the electrodes on both sides of the quartz plate, which is equal to the thickness of the plate and the velocity of the acoustic wave within the quartz material. In other words, electromagnetic energy is converted into acoustic energy, whereby piezoelectricity is associated with the electrical polarization of materials with anisotropic crystal structure. The most applied technique for monitoring the acoustic wave operation is the oscillation method. This means a configuration in which the device constitutes the frequency-controlling element of a circuit. The oscillation method measures the series resonant frequency of the resonating sensor.

The microgravimetric sensor devices are divided into quartz crystal microbalance (QCM) devices applying a thickness-shear mode (TSM), and devices applying a surface acoustic wave (SAW) detection principle. These sensors have reached considerable technical sophistication.

Additional bioanalytical application devices include the flexural plate wave (FPW), the shear horizontal acoustic plate (SH-APM), the surface transverse wave (STW) and the thin-rod acoustic wave (TRAW)

There are considerable similarities between the physical principles of QCM and SPR sensors, even when there are fundamental differences. Both QCM and SPR are wave-propagation phenomena and show resonance structure. The elastic QCM wave and the surface plasmon wave are nonradiative, i.e., an evanescent wave exists. Changes of physical properties within the evanescent field lead to a shift of resonance. Thus, a linear approximation of the physical relationship is allowed for immunological application in immunosensors.

The TSM Sensor

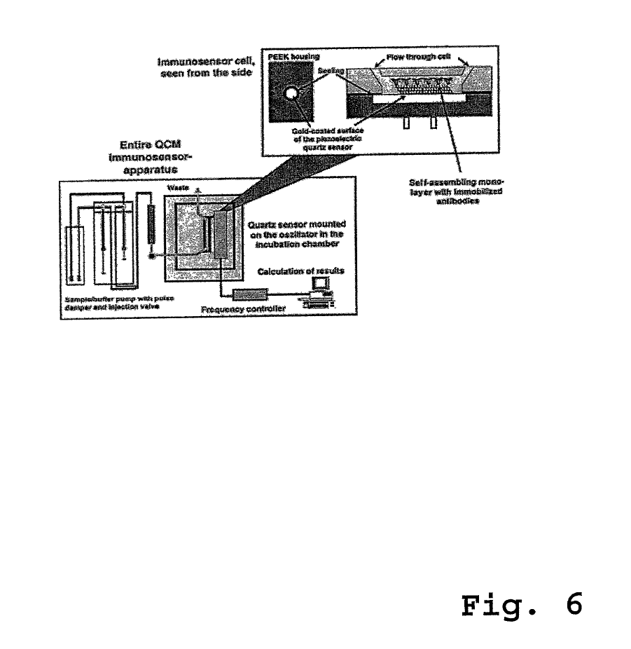

The TSM sensor consists of an AT-cut piezoelectric crystal disc, most commonly of quartz because of its chemical stability in biological fluids and resistance to extreme temperatures. The disc is attached to two metal electrodes on opposite sides for the application of the oscillating electric field. The TSM is run in a range of 5-20 MHz. The schematic design of a typical TSM device shown in FIG. 6. Advantages are, besides the chemical inertness, the low cost of the devices and the reliable quality of the mass-produced quartz discs. Major drawbacks of the system are the insensitivity for analytes with a molecular weight −1000 Da, and, as seen in all label-free immunosensor systems, nonspecific binding interferences. Nonspecific binding effects are hard to distinguish from authentic binding events due to the fact that no reference line can be placed in the sensor device. For a SH-APM device, however, by appropriately selecting the device frequency, these spurious responses can be suppressed. This sensor is applicable for measurements in human serum matrix.

One of the first applications of TSM technology was an immunosensor for human immunodeficiency virus (HIV) serology. This sensor was realized by immobilizing recombinant viral peptides on the surface of the transducer and by detecting anti-HIV antibodies directly in human sera.

The Saw Sensor

SAW sensors use thick ST-cut quartz discs and interdigitated metal electrode arrays that generate acoustic Rayleigh waves in both directions from the interdigital electrodes, their transmission being attenuated by surface-attached biomolecules. The oscillation frequency of a SAW sensor ranges from 30 to 500 MHz. The operation of SAW immunosensors with biological samples is compromised by the fact that the surface wave is considerably attenuated in the liquid phase. Thus, the domain of this technique is most likely restricted to gas phase operations.

The present invention is exemplified as an ELISA as described hereinbelow for corresponding probe substrate and or metabolites and the molar ratios thereof calculated to reveal the individual phenotype.

In Example I, a detailed description of the synthesis of probe substrate and metabolite derivatives and the ELISA development for N-acetyltransferase (NAT2). The materials and methods, and the overall general process described for the development of the NAT2 ELISA method and kit for metabolic phenotyping can be and will be applied to the development of the metabolic phenotyping ELISA kits CYP2C19.

### EXAMPLE I

### Determination of Phenotypic Determinants by ELISA

NAT2

Different probe substrates can be used to determine the NAT2 phenotype (Kilbane, A. J. et al. (1990) Clin. Pharmacol. Ther., 47:470-477; Tang, B-K. et al. (1991) Clin. Pharmacol. Ther., 49:648-657). In accordance with the present invention caffeine is the preferred probe because it is widely consumed and relatively safe (Kalow, W. et al. (1993) Clin. Pharmacol. Ther., 53:503-514). In studies involving this probe, the phenotype has been generally determined from ratios of the caffeine metabolites 5-acetamino-6-amino-1-methyluracil (AAMU) or 5-acetamino-6-formylamino-1-methyluracil (AFMU) and 1-methylxanthine (1X). In these studies, the subjects are given an oral dose of a caffeine-containing substance, and the urinary concentrations of the target metabolites determined by HPLC (Kilbane, A. J. et al. (1990) Clin. Pharmacol. Ther., 47:470-477; Tang, B-K. et al. (1991) Clin. Pharmacol. Ther., 49:648-657) or CE (Lloyd, D. et al. (1992) J. Chrom., 578:283-291).

The number of clinical protocols requiring the determination of NAT2 phenotypes is rapidly increasing and in accordance with the present invention, an enzyme linked immunosorbent assay (ELISA) was developed for use in these studies (Wong, P., Leyland-Jones, B., and Wainer, I. W. (1995) J. Pharm. Biomed. Anal., 13:1079-1086). ELISAs have been successfully applied in the determination of low amounts of drugs and other antigenic components in plasma and urine samples, involve no extraction steps and are simple to carry out.

In accordance with the present invention, antibodies were raised in animals against two caffeine metabolites [5-acetamino-6-amino-1-methyluracil (AAMU) or 5-acetamino-6-formylamino-1-methyluracil (AFMU), and 1-methyl xanthine (1X)] present in urine samples of an individual collected after drinking coffee. Their ratio provides a determination of an individual's N-acetylation (NAT2) phenotype. Subsequently, there was developed a competitive antigen enzyme linked immunosorbent assay (ELISA) for measuring this ratio using these antibodies.

The antibodies of the present invention can be either polyclonal antibodies or monoclonal antibodies raised against two different metabolites of caffeine, which allow the measurement of the molar ratio of these metabolites.

In accordance with the present invention, the molar ratio of caffeine metabolites is used to determine the acetylation phenotype of the individual as follows. Individuals with a ratio less than 1.80 are slow acetylators.

Materials and Methods

Materials

Cyanomethylester, isobutyl chloroformate, dimethylsulfate, sodium methoxide, 95% pure, and tributylamine were purchased from Aldrich (Milwaukee, Wis., USA); horse radish peroxidase was purchased from Boehringer Mannheim (Montreal, Que., Canada); Corning easy wash polystyrene microtiter plates were bought from Canlab (Montreal, Que., Canada); o-methylisourea hydrochloride was obtained from Lancaster Laboratories (Windham, N.H., USA); alkaline phosphatase conjugated to goat anti-rabbit IgGs was from Pierce Chemical Co. (Rockford, Ill., USA); bovine serum albumin fraction V initial fractionation by cold alcohol precipitation (BSA), complete and incomplete Freund's adjuvants, diethanolamine, 1-methylxanthine, p-nitrophenol phosphate disodium salt, o-phenylenediamine hydrochloride; porcine skin gelatin, rabbit serum albumin (RSA); Sephadex™ G25 fine, Tween™ 20 and ligands used for testing antibodies' cross reactivities were obtained from Sigma Chemical Co. (St. Louis, Mo., USA). Whatman™ DE52 diethylaminoethyl-cellulose was obtained from Chromatographic Specialties Inc. (Brockville, Ont., Canada). Dioxane was obtained from A&C American Chemicals Ltd. (Montreal, Que., Canada) and was refluxed over calcium hydride for 4 hours and distilled before use. Other reagents used were of analytical grade.

Synthetic Procedures

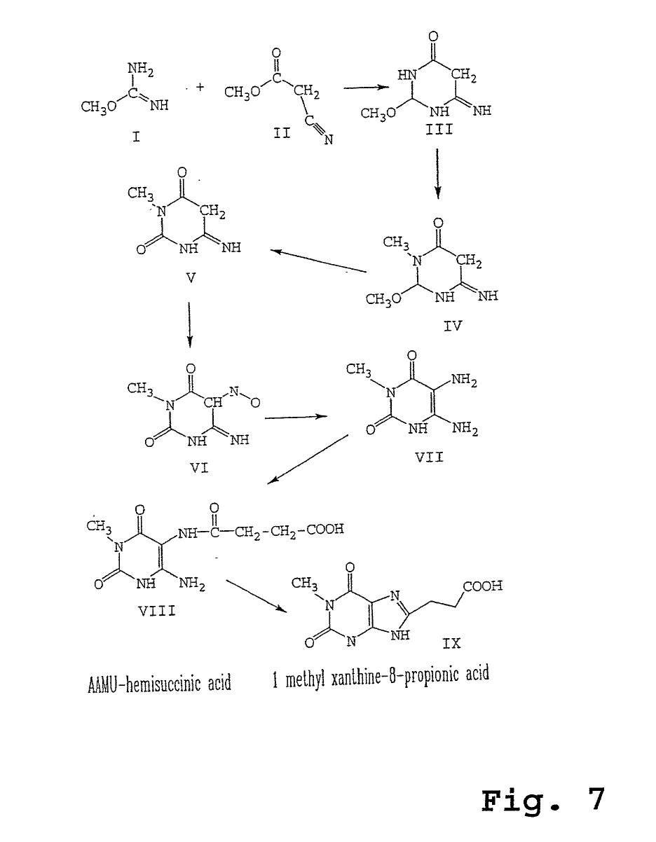

The synthetic route for the production of AAMU-hemisuccinic acid (VIII) and 1-methylxanthine-8-propionic acid (IX) is presented in FIG. 7.

Synthesis of 2-Methoxy-4-Imino-6-Oxo-Dihydropyridine (III)

Compound III is synthesized according to the procedure of Pfeiderer (Pfeilderer, W. (1957) Chem. Ber., 90:2272-2276) as follows. To a 250 mL round bottom flask 12.2 g of o-methylisourea hydrochloride (110.6 mmol), 11.81 mL methylcyanoacetate (134 mmol), 12.45 g of sodium methoxide (230.5 mmol) and 80 mL of methanol are added. The suspension is stirred and refluxed for 5 hours at 68-70° C. After cooling at room temperature, the suspension is filtered through a sintered glass funnel (Pyrex, 40-60 ASTM, 60 mL), and the NaCl on the filter is washed with methanol. The filtrate is filtered by gravity through a Whatman™ no. 1 paper in a 500 mL round bottom flask, and the solvent is evaporated under reduced pressure with a rotary evaporator at 50° C. The residue is solubilized with warm distilled water, and the product is precipitated by acidification to pH 3-4 with glacial acetic acid. After 2 hours (or overnight) at room temperature, the suspension is filtered under vacuum through a sintered glass funnel (Pyrex, 40-60 ASTM, 60 mL). The product is washed with water, acetone, and dried. The product is recrystallized with water as the solvent and using charcoal for decolorizing (activated carbon, Norit^{r} A<100 mesh, decolorizing). The yield is 76%.

Synthesis of 1-Methyl-2-Methoxy-4-Imino-6-Oxo-Dyhydropyrimidine (IV)

Compound IV is synthesized according to the procedure of Pfeiderer (Pfeilderer, W. (1957) Chem. Ber., 90:2272-2276) as follows. To a 250 mL round bottom flask 11 g of compound III (77.0 mmol) and 117 mL of 1N NaOH (freshly prepared) are added. The solution is stirred and cooled at 15° C., using a water bath and crushed ice. Then 11.7 mL dimethylsulfate (123.6 mmol) are added dropwise with a pasteur pipette over a period of 60 min. Precipitation eventually occurs upon the addition. The suspension is stirred at 15° C. for 3 hours and is left at 4° C. overnight. The product is recovered by filtration under vacuum through a sintered glass funnel (Pyrex, 40-60 ASTM, 60 mL). The yield is 38%.

Synthesis of 1-Methyl-4-Iminouracil (V)

Compound V is synthesized according to the procedure of Pfeiderer (Pfeilderer, W. (1957) Chem. Ber., 90:2272-2276) as follows. To a 250 mL round bottom flask 11.26 g of compound IV (72.6 mmol) and 138 mL 12 N HCl are added, and the suspension is stirred at room temperature for 16-20 hours. The suspension is cooled on crushed ice, the product is recovered by filtration under vacuum through a sintered glass funnel (Pyrex, 40-60 ASTM, 60 mL). The product is washed with water at 4° C., using a pasteur pipette, until the pH of filtrate is around 4 (about 150 mL). The product is washed with acetone and dried. The yield is 73%.

Synthesis of 1-Methyl-4-Imino-5-Nitrouracil (VI)

Compound VI is synthesized according to the procedure of Lespagnol et al (Lespagnol, A. et al. (1970) Chim. Ther., 5:321-326) as follows. To a 250 mL round bottom flask 6.5 g of compound V (46 mmol) and 70 mL of water are added. The suspension is stirred and refluxed at 100° C. A solution of 6.5 g sodium nitrite (93.6 mmol) dissolved in 10 mL water is added gradually to the reaction mixture with a pasteur pipette. Then 48 mL of glacial acetic acid is added with a pasteur pipette. Upon addition, precipitation occurs and the suspension becomes purple. The suspension is stirred and heated for an additional 5 min., and cooled at room temperature and then on crushed ice. The product is recovered by filtration under vacuum through a sintered glass funnel (Pyrex, 10-15 ASTM, 60 mL). It is washed with water at 4° C. to remove acetic acid and then with acetone. Last traces of acetic acid and acetone are removed under a high vacuum. The yield is 59%.

Synthesis of 1-Methyl-4,5-Diaminouracil (VII)

Compound VII is synthesized by the procedure of Lespagnol et al. (Lespagnol, A. et al. (1970) Chim. Ther., 5:321-326) as follows. To a 100 mL round bottom flask 2 g of compound VI (11.7 mmol) and 25 mL water are added. The suspension is stirred and heated in an oil bath at 60° C. Sodium hydrosulfite (88%) is gradually added (40.4 mmol), using a spatula, until the purple color disappears (approximately 5 g or 24.3 mmol) The suspension is heated for an additional 15 min. The suspension is cooled on crushed ice and left at 4° C. overnight. The product is recovered by filtration under vacuum through a sintered glass funnel (Pyrex, 30-40 ASTM, 15 mL). The product is washed with water and acetone, and dried. The last traces of acetone are removed under a high vacuum. The yield is 59%.

Synthesis of AAMU-Hemisuccinic Acid (VIII)

Compound VIII is synthesized as follows. To a 20 mL beaker 0.30 g of compound VII (1.92 mmol) and 5 mL water are added. The suspension is stirred and the pH is adjusted between 8 to 9 with a 3N NaOH solution. Then 0.33 g succinic anhydride (3.3 mmol) is added to the resulting solution, and the mixture is stirred until the succinic anhydride is dissolved. During this process, the pH of the solution is maintained between 8 and 9. The reaction is completed when all the succinic anhydride is dissolved and the pH remains above 8. The hemisuccinate is precipitated by acidification to pH 0.5 with 12N HCl. The product is recovered by filtration on a Whatman™ No. 1 paper, and washed with water to remove HCl. It is then washed with acetone and dried.

Other AAMU or AFMU Derivatives

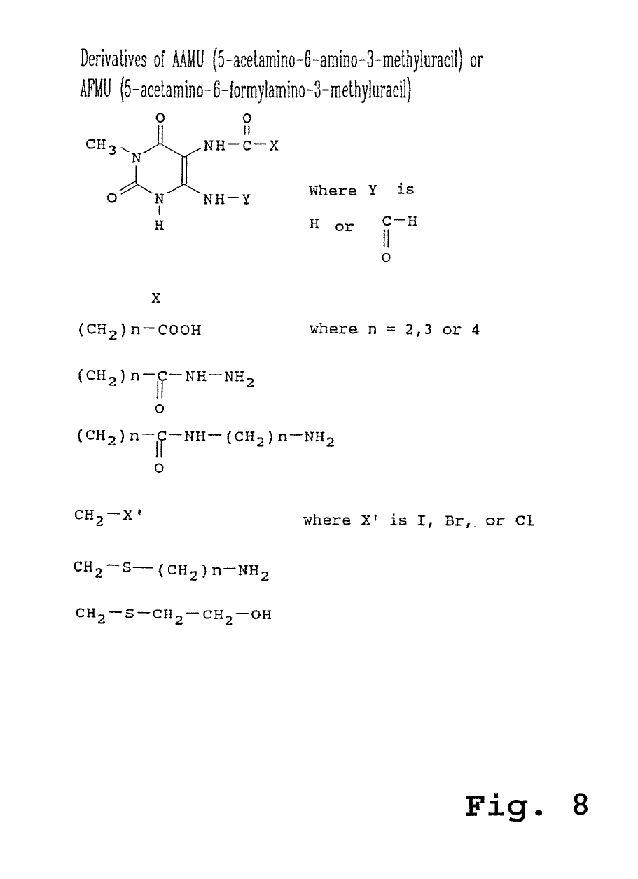

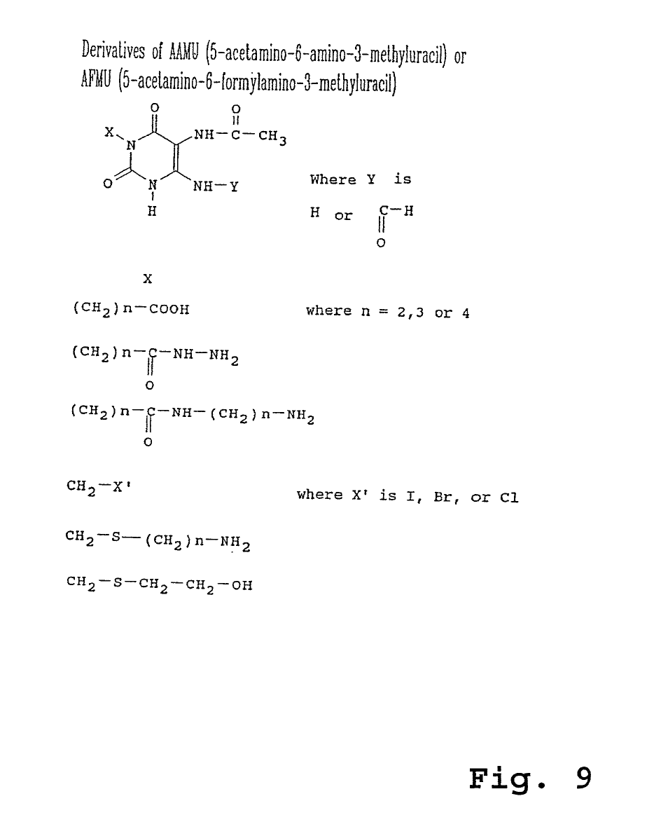

The derivatives shown in FIGS. 8 and 9 can also be used for raising antibodies against AAMU or AFMU that can be used for measuring the concentrations of these caffeine metabolites in urine samples.

Synthesis of 1-Methylxanthine-8-Propionic Acid (IX)

This product is synthesized according to a modified procedure of Lespagnol et al. (Lespagnol, A. et al. (1970) Chim. Ther., 5:321-326) as follows. A 0.2 g sample of compound VIII (0.78 mmol) is dissolved in 2-3 mL of a 15% NaOH solution. The resulting solution is stirred at 100° C. until all of the solvent is evaporated, and is then maintained at this temperature for an additional 5 min. The resulting solid is cooled at room temperature, and dissolved in 10 mL water. The product is precipitated by acidification to pH 2.8 with 12 N HCl. After cooling at 4° C. for 2.5 hours, the product is recovered by filtration on a Whatman™ No. 1 paper, washed with water and acetone, and dried. It is recrystallized from water-methanol (20:80, v/v), using charcoal to decolorize the solution.

Other Derivatives of 1X

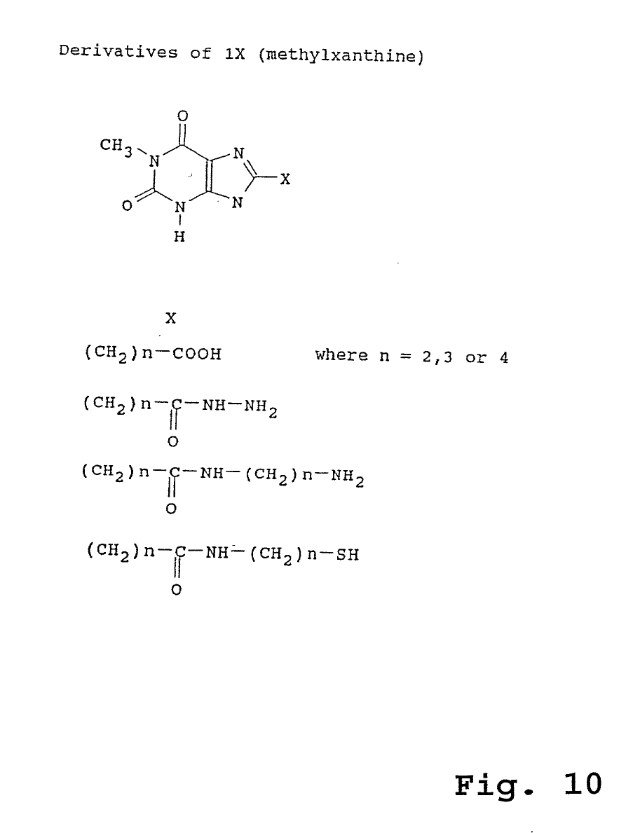

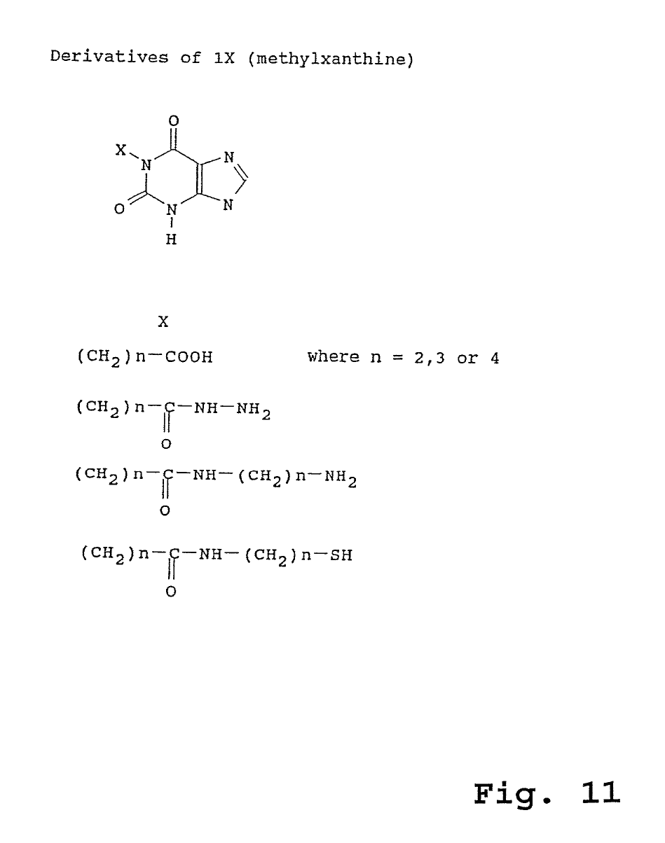

The other derivatives of 1X, shown in FIGS. 10 and 11, can also be used for raising antibodies against 1X and thereby to allow the development of an ELISA for measuring 1X concentration in urine samples.

Synthesis of AAMU

AAMU is synthesized from compound VII according to the procedure of Fink et al (Fink, K. et al. (1964) J. Biol. Chem., 249:4250-4256) as follows. To a 100 mL round bottom flask 1.08 g of compound VII (6.9 mmol) and 20 mL acetic acid anhydride were added. The suspension is stirred and refluxed a 160-165° C. for 6 min. After cooling at room temperature, the suspension is filtered under vacuum through a sintered glass funnel (Pyrex, 10-15 ASTM, 15 mL). The product is washed with water and acetone, and dried. The product is recrystallized in water.

NMR Spectroscopy

¹H and ¹³C NMR spectra of compounds VIII and IX are obtained using a 500 MHz spectrophotometer (Varian™ XL 500 MHz, Varian Analytical Instruments, San Fernando, Calif., USA) using deuterated dimethyl sulfoxide as solvent.

Conjugation of Haptens to Bovine Serum Albumin and Rabbit Serum Albumin

The AAMU-hemisuccinic acid (VIII) and the 1-methylxanthine propionic acid (IX) are conjugated to BSA and RSA according to the following mixed anhydride method. To a 5 mL round bottom flask 31.7 mg of compound VIII (0.12 mmol) or 14.9 mg of compound IX (0.06 mmol) are added. Then 52.2 μL of tri-n-butylamine (0.24 mmol) and 900 μL of dioxane, dried over calcium hydride and freshly distilled, are added. The solution is cooled at 10° C. in a water bath using crushed ice. Then 12.6 μL isobutyl chloroformate at 4° C. (0.12 mmol, recently purchased or opened) are added and the solution is stirred for 30-40 min at 10-12° C. While the above solution is stirring, a second solution is prepared as follows. In a glass tube 70 mg BSA or RSA (0.001 mmol) are dissolved in 1.83 mL water. Then 1.23 mL dioxane, freshly dried and distilled, is added and the BSA or RSA solution is cooled on ice. After 30-40 min of the above stirring, 70 μL of 1 N NaOH solution cooled on ice is added to the BSA or RSA solution and the resulting solution is poured in one portion to the flask containing the first solution. The solution is stirred at 10-12° C. for 3 hours and dialyzed against 1 liter of water for 2 days at room temperature, with water changed twice a day. The protein concentration of the conjugates and the amounts of moles of AAMU or 1X incorporated per mole of BSA or RSA is determined by methods described below. The products are stored as 1 mL aliquots at −20° C.

Protein Determination by the Method of Lowry et al. (Lowry, O. H. et al. (1951) J. Biol. Chem., 193:265-275)

A) Solutions

Solution A: 2 g Na₂CO₃ is dissolved in 50 mL water, 10 mL of 10% SDS and 10 mL 1 NaOH, water is added to 100 mL. Freshly prepared.

Solution B: 1% NaK Tartrate

Solution C: 1% CuSO₄.5H₂O

Solution D: 1 N phenol (freshly prepared): 3 mL Folin & Ciocalteu's phenol reagent (2.0 N) and 3 mL water.

Solution F: 98 mL Solution A, 1 mL Solution B, 1 mL

Solution C. Freshly prepared.

BSA: 1 mg/mL. 0.10 g bovine serum albumin (fraction V)/100 mL.

B) Assay

| Standard curve | Tubes # (13 × 100 mm) | | | | | | |
|---|---|---|---|---|---|---|---|
| Solution | 1 | 2 | 3 | 4 | 5 | 6 | 7 |
| BSA μL) | 0 | 10 | 15 | 20 | 30 | 40 | 50 |
| Water μL) | 200 | 190 | 185 | 180 | 170 | 160 | 150 |
| Solution F (mL) | 2.0 | 2.0 | 2.0 | 2.0 | 2.0 | 2.0 | 2.0 |

The solutions are vortexed and left 10 min at room temperature.

| | | | | | | | |
|---|---|---|---|---|---|---|---|
| Solution D μL) | 200 | 200 | 200 | 200 | 200 | 200 | 200 |

The solutions are vortexed and left at room temperature for 1 hour.

The absorbance of each solution is read at 750 nm using water as the blank.

UNKNOWN

| | Tube # (13 × 100 mm) | | |
|---|---|---|---|
| Solution D.F.^{a} | 1 | 2 | 3 |
| Unknown (μL) | x | x | x |
| Water (μL) | y | y | y |
| | x + y = 200 μL | | |
| Solution F (mL) | 2.0 | 2.0 | 2.0 |
| The solutions are vortexed and left 10 min at room | | | |
| temperature. | | | |
| Solution D (μL) | 200 | 200 | 200 |

The solutions are vortexed and left 1 hour at room temperature.

The absorbance of each solution is read at 750 nm using water as the blank.

The protein concentration is calculated using the standard curve and taking account of the dilution factor (D.F.).

a. D.F. (dilution factor). It has to be such so that the absorbance of the unknown at 750 nm is within the range of absorbance of the standards.

Method to Determine the Amounts of Moles of AAMU or 1X Incorporated Per Mole of BSA or RSA

This method gives an approximate estimate. It is a useful one because it allows one to determine whether the coupling proceeded as expected.

A) Solutions

10% sodium dodecyl sulfate (SDS)

1% SDS solution

0.5 or 1 mg/mL of AAMU-BSA (or AAMU-RSA) in a 1% SDS solution (1 mL).

0.5 or 1 mg/mL of BSA or RSA in a 1% SDS solution (1 mL).

B) Procedure

The absorbance of the AAMU conjugate solution is measured at 265 nm, with 1% SDS solution as the blank.

The absorbance of the BSA (or RSA) solution is measured at 265 nm, with 1% SDS solution as the blank.

The amount of moles of AAMU incorporated per mole of BSA (or RSA) is calculated with this formula:

$$y = \frac{{A}_{265} ( AAMU - BSA ) - {A}_{265} ( BSA )}{{ϵ}_{265} ( AAMU ) \times [ BSA ]}$$

Where:

y is the amount of moles of AAMU/mole of BSA (or RSA);

ε₂₆₅ (AAMU) is the extinction coefficient of AAMU=10⁴ M⁻¹cm⁻¹; and

[BSA]=BSA (mg/mL)/68,000/mmole.

To calculate the amount of moles of 1X incorporated per mole of BSA or RSA, the same procedure is used but with this formula:

$$y = \frac{{A}_{252} ( 1 X - BSA ) - {A}_{252} ( BSA )}{{ϵ}_{252} ( 1 X ) \times [ BSA ]}$$

Where:

y is the amount of moles of 1X/mole of BSA (or RSA);

ε₂₅₂ (AAMU) is the extinction coefficient of 1X=10⁴ M⁻¹cm⁻¹; and

[BSA]=BSA (mg/mL)/68,000/mmole.

Coupling of Haptens to Horse Radish Peroxidase

The AAMU derivative (VIII) and 1X derivative (IX) are conjugated to horse radish peroxidase (HRP) by the following procedure. To a 5 mL round bottom flask 31.2 mg of compound VIII (or 28.3 mg of compound IX) are added. Then 500 μL of dioxane, freshly dried over calcium chloride, are added. The suspension is stirred and cooled at 10° C. using a water bath and crushed ice. Then 114 μL tributylamine and 31 μL of isobutyl chloroformate (recently opened or purchased) are added. The suspension is stirred for 30 min at 10° C. While the suspension is stirring, a solution is prepared by dissolving 13 mg of horse radish peroxidase (HRP) in 2 mL of water. The solution is cooled at 4° C. on crushed ice. After the 30 min stirring, 100 μL of a 1 N NaOH solution at 4° C. is added to the HRP solution and the alkaline HRP solution is poured at once into the 5 mL flask. The suspension is stirred for 4 hours at 10-12° C. The free derivative is separated from the HRP conjugate by filtration through a Sephadex G-25™ column (1.6×30 cm) equilibrated and eluted with a 0.05 M sodium phosphate buffer, pH 7.5. The fractions of 1.0-1.2 mL are collected with a fraction collector. During the elution two bands are observed: the HRP conjugate band and a light yellow band behind the HRP conjugate band. The HRP conjugate elutes between fractions 11-16. The fractions containing the HRP conjugate are pooled in a 15 mL tissue culture tube with a screw cap. The HRP conjugate concentration is determined at 403 nm after diluting an aliquot (usually 50 μL+650 μL of buffer).

[HRP-conjugate](mg/mL)=A₄₀₃×0.4×D.F.

The ultraviolet (UV) absorption spectrum is recorded between 320 and 220 nm. The presence of peaks at 264 and 270 nm for AAMU-HRP and 1X-HRP conjugates, respectively, are indicative that the couplings proceeded as expected.

After the above measurements, 5 μL of a 4% thiomersal solution is added per mL of the AAMU-HRP or 1X-HRP conjugate solution. The conjugates are stored at 4° C.

Antibody Production

Four mature females New Zealand white rabbits (Charles River Canada, St-Constant, Que., Canada) are used for antibody production. The protocol employed in this study was approved by the McGill University Animal Care Committee in accordance with the guidelines from the Canadian Council on Animal Care. Antibodies of the present invention may be monoclonal or polyclonal antibodies.

An isotonic saline solution (0.6 mL) containing 240 mg of BSA conjugated antigen is emulsified with 0.6 mL of a complete Freund's adjuvant. A 0.5 mL aliquot of the emulsion (100 mg of antigen) is injected per rabbit intramuscularly or subcutaneously. Rabbits are subsequently boosted at intervals of three weeks with 50 mg of antigen emulsified in incomplete Freund's adjuvant. Blood is collected by venipuncture of the ear 10-14 days after boosting. Antisera are stored at 4° C. in the presence of 0.01% sodium azide.

Double Immunodiffusion in Agar Plate

An 0.8% agar gel in PBS is prepared in a 60×15 mm petri dish. Rabbit serum albumin (100 μL of 1 mg mL⁻¹) conjugated to AAMU (or 1X) are added to the center well, and 100 μL of rabbit antiserum are added to the peripheral wells. The immunodiffusion is carried out in a humidified chamber at 37° C. overnight and the gel is inspected visually.

Antiserum Titers

The wells of a microtiter plate are coated with 10 μg mL⁻¹ of rabbit serum albumin-AAMU (or 1X) conjugate in sodium carbonate buffer, pH 9.6) for 1 hour at 37° C. (100 μL/per well). The wells are then washed three times with 100 μL TPBS (phosphate buffer saline containing 0.05% Tween™ 20) and unoccupied sites are blocked by an incubation with 100 mL of TPBS containing 0.05% gelatin for 1 hour at 37° C. The wells are washed three times with 100 μL TPBS and 100 μL of antiserum diluted in TPBS are added. After 1 hour at 37° C., the wells are washed three times with TPBS, and 100 μL of goat anti-rabbit IgGs-alkaline phosphatase conjugate, diluted in PBS containing 1% BSA, are added. After 1 hour at 37° C., the wells are washed three times with TPBS and three times with water. To the wells are added 100 μL of a solution containing MgCl₂ (0.5 mM) and p-nitrophenol phosphate (3.85 mM) in diethanolamine buffer (10 mM, pH 9.8). After 30 min. at room temperature, the absorbency is read at 405 nm with a microplate reader. The antibody titer is defined as the dilution required to change the absorbance by one unit (1 au).

Isolation of Rabbit IgGS

The DE52-cellulose resin is washed three times with sodium phosphate buffer (500 mM, pH 7.50), the fines are removed and the resin is equilibrated with a sodium phosphate buffer (10 mM, pH 7.50). The resin is packed in a 50×1.6 cm column and eluted with 200-300 mL equilibrating buffer before use. To antiserum obtained from 50 mL of blood (30-32 mL) is added dropwise 25-27 mL of a 100% saturated ammonium sulfate solution with a Pasteur pipette. The suspension is left at room temperature for 3 h and centrifuged for 30 min. at 2560 g at 20° C. The pellet is dissolved with 15 mL sodium phosphate buffer (10 mM, pH 7.50) and dialyzed at room temperature with the buffer changed twice per day. The dialyzed solution is centrifuged at 2560 g for 10 min. at 20° C. to remove precipitate formed during dialysis. The supernatant is applied to the ion-exchange column. Fractions of 7 mL are collected. After application, the column is eluted with the equilibrating buffer until the absorbance at 280 nm becomes less than 0.05 au. The column is then eluted with the equilibrating buffer containing 50 mM NaCl. Fractions having absorbencies greater than 0.2 at 280 nm are saved and stored at 4° C. Protein concentrations of the fractions are determined as described above.

Competitive Antigen ELISA

Buffers and water without additives are filtered through millipore filters and kept for 1 week. BSA, antibodies, Tween™ 20 and horse radish peroxidase conjugates are added to these buffers and water just prior to use. Urine samples are usually collected 4 hours after drinking a cup of coffee (instant or brewed with approximately 100 mg of caffeine per cup) and stored at −80° C. The urine samples are diluted 10 times with sodium phosphate buffer (620 mosm, pH 7.50) and are subsequently diluted with water to give concentrations of AAMU and 1X no higher than 3×10⁻⁶ M in the ELISA. All the pipettings are done with an eight-channel pipette, except those of the antibody and sample solutions. Starting with the last well, 100 μL of a carbonate buffer (100 mM, pH 9.6) containing 2.5 μg mL-¹ antibodies are added to each well. After 90 min. at room temperature, the wells are washed three times with 100 mL of TPB: isotonic sodium phosphate buffer (310 mosm, pH 7.50) containing 0.05% Tween™ 20.

After the initial wash, unoccupied sites are blocked by incubation for 90 min. at room temperature with 100 μL TBP containing 3% BSA. The wells are washed four times with 100 μL TPB. The washing is followed by additions of 50 μL of 12 mg mL⁻¹ AAMU-HRP or 1X-HRP conjugate in 2×TPB containing 2% BSA, and 50 μL of either water, standard (13 standards; AAMU or 1X, 2×10⁻⁴ to 2×10⁻⁸ M) or sample in duplicate. The microplate is gently shaken with an orbital shaker at room temperature for 3-4 hours. The wells are washed three times with 100 μL TPB containing 1% BSA and three times with water containing 0.05% Tween™ 20. To the washed plate is added 150 μL of a substrate buffer composed of citric acid (25 mM) and sodium phosphate dibasic buffer (50 mM, pH 5.0) containing 0.06% hydrogen peroxide and 0.04% o-phenylenediamine hydrochloride. After 20 min. at room temperature with shaking, the reaction is stopped with 50 μL of 2.5 M HCl. After shaking the plate 3 min., the absorbances are read with a microtiter plate reader at 490 nm.

Results

Polyclonal antibodies against AAMU and 1X could be successfully raised in rabbits after their conjugation to bovine serum albumin. Each rabbit produced antibody titers of 30,000-100,000 as determined by ELISA. This was also indicated by strong precipitation lines after double immunodiffusion in agar plates of antisera and derivatives conjugated to rabbit serum albumin. On this basis, a) IgGs antibodies were isolated on a DE-52 cellulose column and b) a competitive antigen ELISA for NAT2 phenotyping using caffeine as probe substrate was developed according to the methods described in the above section entitled Materials and Methods.

Contrary to current methods used for phenotyping, the assay involves no extraction, is sensitive and rapid, and can be readily carried out on a routine basis by a technician with a minimum of training in a clinical laboratory.

The present invention will be more readily understood by referring to the following examples which are given to illustrate the invention rather than to limit its scope.

A Competitive Antigen ELISA for NAT2 Phenotyping Using Caffeine as a Probe Substrate

Buffers and water without additives were filtered through millipore filters and kept for 1 week. BSA, antibodies, Tween™ 20 and horse radish peroxidase conjugates were added to these buffers and water just prior to use. Urine samples were usually collected 4 hours after drinking a cup of coffee (instant or brewed with approximately 100 mg of caffeine per cup) and stored at −80° C. They were diluted 10 times with sodium phosphate buffer (620 mosm, pH 7.50) and were subsequently diluted with water to give concentrations of AAMU and 1X no higher than 3×10⁻⁶ M in the ELISA. All the pipettings were done with an eight-channel pipette, except those of the antibody and sample solutions. Starting with the last well, 100 μL of a carbonate buffer (100 mM, pH 9.6) containing 2.5 μg mL-¹ antibodies was pipetted. After 90 min. at room temperature, the wells were washed three times with 100 μL of TPB: isotonic sodium phosphate buffer (310 mosm, pH 7.50) containing 0.05% Tween™ 20.

After the initial wash, unoccupied sites were blocked by incubation for 90 min. at room temperature with 100 μL TBP containing 3% BSA. The wells were washed four times with 100 μL TPB. This was followed by additions of 50 μL of 12 mg mL⁻¹ AAMU-HRP or 1X-HRP conjugate in 2×TPB containing 2% BSA, and 50 μL of either water, standard (13 standards; AAMU or 1X, 2×10⁻⁴ to 2×10⁻⁸ M) or sample in duplicate. The microplate was gently shaken with an orbital shaker at room temperature for 3-4 hours. The wells were washed three times with 100 μL with TPB containing 1% BSA and three times with water containing 0.05% Tween™ 20. To the washed plate was added 150 μL of a substrate buffer composed of citric acid (25 mM) and sodium phosphate dibasic buffer (50 mM, pH 5.0) containing 0.06% hydrogen peroxide and 0.04% o-phenylenediamine hydrochloride. After 20 min. at room temperature with shaking, the reaction was stopped with 50 μL of 2.5 N HCl. After shaking the plate 3 min., the absorbances were read with a microtiter plate reader at 490 nm.

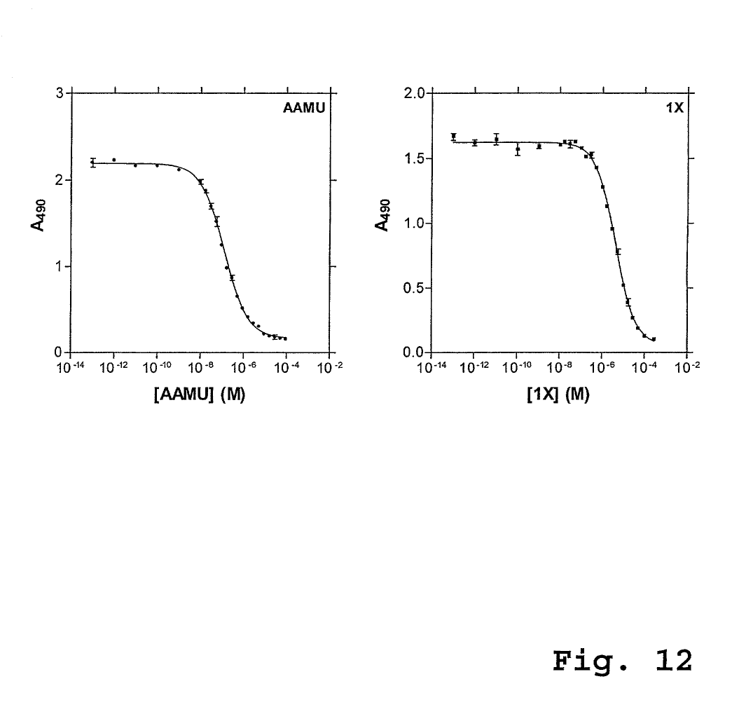

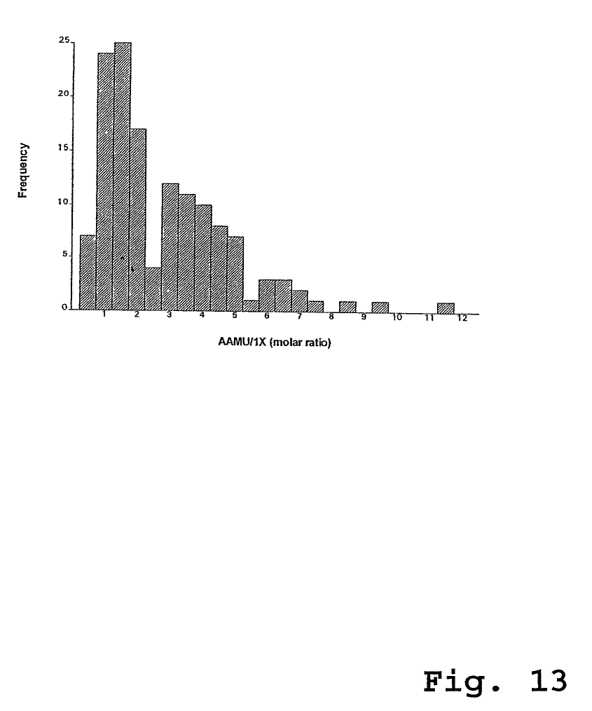

The competitive antigen ELISA curves of AAMU-Ab and 1X-Ab determinations obtained in duplicate are presented in FIG. 12. Each calibration curve represents the average of two calibration curves. The height of the bars measure the deviations of the absorbency values between the two calibration curves. Data points without bars indicate that deviations of the absorbency values are equal or less than the size of the symbols representing the data points. Under the experimental conditions of the ELISA: background was less than 0.10 au; the practical limits of detection of AAMU and 1X were 2×10⁻⁷ M and 2×10⁻⁶ M, respectively, concentrations 500 and 50 times lower than those in urine samples from previous phenotyping studies (Kilbane, A. J. et al. (1990) Clin. Pharmacol. Ther., 47:470-477); the intra-assay and interassay coefficients of variations of AAMU and 1X were 15-20% over the concentration range of 0.01-0.05 mM.

A variety of conditions for the ELISA were tested and a number of noteworthy observations were made: gelatin, which was used in the competitive antigen ELISA determination of caffeine in plasma (Fickling, S. A. et al. (1990) J. Immunol. Meth., 129:159-164), could not be used in our ELISA owing to excessive background absorbency which varied between 0.5 and 1.0 au; in the absence of Tween™ 20, absorbency changes per 15 min. decreased by a factor of at least 3, and calibration curves were generally erratic; absorbency coefficients of variation of samples increased by a factor of 3 to 4 when the conjugates and haptens were added to the wells as a mixture instead individually.

The cross reactivities of AAMU-Ab and 1X-Ab were tested using a wide variety of caffeine metabolites and structural analogs (Table 2 below). AAMU-Ab appeared highly specific for binding AAMU, while 1X-Ab appeared relatively specific for binding 1X. However, a 11% cross reactivity was observed with 1-methyluric acid (1U), a major caffeine metabolite.

TABLE 2
Cross-reactivity of AAMU-Ab and 1X-Ab towards different caffeine
metabolites and structural analogs

| | % Cross-Reaction | |
|---|---|---|
| Compound | AAMU-Ab | 1X-Ab |
| Xanthine | 0^{a} | 0 |
| Hypoxanthine | 0 | 0 |
| 1-Methyl Xanthine (1X) | 0 | 100 |
| 3-Methyl Xanthine | 0 | 0 |
| 7-Methyl Xanthine | 0 | 0 |
| 8-Methyl Xanthine | 0 | 0 |
| 1,3-Dimethyl Xanthine (Theophylline) | 0 | 0.2 |
| 1,7-Dimethyl Xanthine (Paraxanthine) | 0 | 0.5 |
| 3,7-Dimethyl Xanthine (Theobromine) | 0 | 0 |
| 1,3,7-Trimethyl Xanthine (Caffeine) | 0 | 0 |
| Uric acid | 0 | 0 |
| 1-Methyluric acid | 0 | 11 |
| 1,7-Dimethyluric acid | 0 | 0 |
| Guanine | 0 | 0 |
| Uracil | 0 | 0 |
| 5-Acetamino-6-amino-uracil | 0.6 | 0 |
| 5-Acetamino-6-amino-1-methyluracil (AAMU) | 100 | 0 |
| 5-Acetamino-6-amino-1,3-dimethyluracil | 0 | 0 |

a. The number 0 indicates either an absence of inhibition or an inhibition no higher than 40% at the highest compound concentration tested in the ELISA (5×10⁻³ M); concentrations of 5-acetamino-6-amino-1-methyluracil (AAMU) and 1-Methyl Xanthine (1X) required for 50% inhibition in the competitive antigen ELISA were 1.5×10⁻⁶ M and 10⁻⁵ M, respectively.

The relative high level of cross reactivity of 1U is, however, unlikely to interfere significantly in the determination of 1X and the assignment of NAT2 phenotypes, since the ratio of 1U:1X is no greater than 2.5:1 in 97% of the population (Tang, B-K. et al. (1991) Clin. Pharmacol. Ther., 49:648-657). This is confirmed by measurements of apparent concentrations of 1X when the ratio varied between 0-8.0 at the fixed 1X concentration of 3×10⁻⁶ M (Table 3 below). At 1U:1X ratios of 2.5 and 3.0, the apparent increases were 22% and 32%, respectively.

TABLE 3
The effect of the ratio 1U:1X on the determination of 1X concentration by
ELISA at fixed 1X concentration of 3 × 10⁶ M

| 1U:1X ratio | [1X] × 10⁶ (M) |
|---|---|
| 0.0 | 3.00 |
| 0.50 | 2.75 |
| 1.00 | 3.25 |
| 1.50 | 3.25 |
| 2.00 | 3.60 |
| 2.50 | 3.65 |
| 3.00 | 3.95 |
| 4.00 | 4.20 |
| 5.00 | 4.30 |
| 6.00 | 4.50 |
| 8.00 | 4.30 |

The following observations attested to the validity of the competitive antigen ELISA for NAT2 phenotyping.

1) The ELISA assigned the correct phenotype in 29 of 30 individuals that have been phenotyped by capillary electrophoresis (CE) (Lloyd, D. et al. (1992) J. Chrom., 578:283-291).

2) In the CE method, the phenotype was determined using AFMU/1X peak height ratios rather than the AAMU/1X molar ratios used in the ELISA. When the molar ratios determined by ELISA and the peak height ratios determined by CE were correlated by regression analysis, the calculated regression equation was y=0.48+0.87 x, with a correlation coefficient (r) of 0.84, Taking account that these two ratios are not exactly equal and that Kalow and Tang (Kalow, W. et al. (1993) Clin. Pharmacol. Ther., 53:503-514) have pointed out that using AFMU rather than AAMU can lead to misclassification of NAT2 phenotypes, there is a remarkable agreement between the two methods.

3) The ELISA was used in determining the NAT2 phenotype distribution within a group of 146 individuals. 13 illustrates a histogram of the NAT2 phenotypes of this group as determined by measuring the AAMU/1X ratio in urine samples by ELISA. Assuming an antimode of 1.80, the test population contained 60.4% slow acetylators and 39.6% fast acetylators. This is consistent with previously reported distributions (Kalow, W. et al. (1993) Clin. Pharmacol. Ther., 53:503-514; Kilbane, A. J. et al. (1990) Clin. Pharmacol. Ther., 47:470-477).

Determination of 5-Acetamino-6-Amino-1-Methyluracyl (AAMU) and 1-Methyl Xanthine in Urine Samples With the ELISA Kit

TABLE 4
Content of the ELISA kit and conditions of storage

| | | | | | Storage |
|---|---|---|---|---|---|
| Item | Unit | State | Amt | | conditions |
| Tween ™ 20 | 1 vial | Liquid | 250 | μL/vial | 4° C. |
| H₂O₂ | 1 vial | Liquid | 250 | μL/vial | 4° C. |
| AAMU-HRP | 1 vial | Liquid | 250 | μL/vial | 4° C. |
| 1X-HRP | 1 vial | Liquid | 250 | μL/vial | 4° C. |
| Buffer A | 4 vials | Solid | 0.8894 | g/vial | 4° C. |
| Buffer B | 6 vials | Solid | 1.234 | g/vial | 4° C. |
| Buffer C | 6 vials | Solid | 1.1170 | g/vial | 4° C. |
| Buffer D | 6 vials | Solid | 0.8082 | g/vial | 4° C. |
| Plate(AAMU- | 2 | Solid | | — | 4° C. |
| Ab) | | | | | |
| Plate (1X-Ab) | 2 | Solid | | — | 4° C. |
| Buffer E | 6 vials | Solid | 0.9567 | g/vial | −20° C. |
| Standards | 14 vials | Liquid | 200 | μ/L | −20° C. |
| (AAMU) | | | | | |
| Standards(1X) | 14 vials | Liquid | 200 | μL | −20° C. |
| 1N NaOH | 1 bottle | Liquid | 15 | mL | 20° C. |
| 1N HCl | 1 bottle | Liquid | 15 | mL | 20° C. |

Conversion of AFMU to AAMU

In order to determine the AAMU concentrations in urine samples by competitive antigen ELISA, a transformation of AFMU to AAMU is required. The contents of an ELISA kit for this assay are listed in Table 4.

Thaw and warm up to room temperature the urine sample.

Suspend the sample thoroughly with the vortex before pipeting.

Add 100 μL of a urine sample to a 1.5 mL-microtube.

Add 100 μL of a 1N NaOH solution.

Leave at room temperature for 10 min.

Neutralize with 100 μL 1N HCl solution.

Add 700 μL of Buffer A (dissolve the powder of one vial A/50 mL).

Dilutions of Urine Samples for the Determinations of [AAMU] and [1X] by ELISA

The dilutions of urine samples required for determinations of AAMU and 1X are a function of the sensitivity of the competitive antigen ELISA and AAMU and 1X concentrations in urine samples. It is suggested to dilute the urine samples by a factor so that AAMU and 1X concentrations are about 3×10⁻⁶ M in the well of the microtiter plate. Generally, dilution factors of 100-400 (Table 5) and 50-100 have been used for AAMU and 1X, respectively.

TABLE 5
Dilution Factors for Identifying AAMU
and 1X Concentrations

| | Microtube # | | | | | | | |
|---|---|---|---|---|---|---|---|---|
| Dilution Factor | 20x | 40x | 50x | 80x | 100x | 150x | 200x | 400x |
| Solution | 1 | 2 | 3 | 4 | 5 | 6 | 7 | 8 |
| Urine | 500 | 250 | 200 | 125 | 100 | 66.7 | 50 | 25 |
| sample (mL)^{a} | | | | | | | | |
| 10 x diluted | | | | | | | | |
| Buffer B (mL) | 500 | 750 | 800 | 875 | 900 | 933.3 | 950 | 975 |

^{a}Vortex the microtubes containing the urine sample before pipeting.
Store the diluted urine sample at −20° C.
Buffer B: dissolve the content of one vial B/100 mL

Determination of [AAMU] and [1 X]In Diluted Urine Samples by ELISA

Precautions

The substrate is carcinogenic. Wear surgical gloves when handling Buffer E (Substrate buffer). Each sample is determined in duplicate. An excellent pipeting technique is required. When this technique is mastered the absorbance values of duplicates should be within less than 5%. Buffers C, D and E are freshly prepared. Buffer E-H₂O₂ is prepared just prior pipeting in the microtiter plate wells.

Preparation of Samples

Prepare Table 6 with a computer and print it. This table shows the content of each well of a 96-well microtiter plate. Enter the name of the urine sample (or number) at the corresponding well positions in Table 6. Select the dilution factor (D.F.) of each urine sample and enter at the corresponding position in Table 6. Enter the dilution of each urine sample with buffer B at the corresponding position in Table 6: for example, for a D.F. of 100 (100 μL of 10× diluted urine sample +900 μL buffer B), enter 100/900. See “Dilutions of urine samples . . . ” procedure described above for the preparation of the different dilutions. Prepare the different dilutions of the urine samples in 1.5-mL microtubes. Prepare Table 7 with a computer and print it. Prepare the following 48 microtubes in the order indicated in Table 7.

TABLE 6
Positions of blanks, control and
urine samples in a microtiter plate

| Sample | Well # | D.F | Dil. |
|---|---|---|---|
| Blank | 1-2 | — | |
| Control | 3-4 | — | |
| S1 | 5-6 | — | |
| S2 | 7-8 | — | |
| S3 | 9-10 | — | |
| S4 | 11-12 | — | |
| S5 | 13-14 | — | |
| S6 | 15-16 | — | |
| S7 | 17-18 | — | |
| S8 | 19-20 | — | |
| S9 | 21-22 | — | |
| S10 | 23-24 | — | |
| S11 | 25-26 | — | |
| S12 | 27-28 | — | |
| S13 | 29-30 | — | |
| S14 | 31-32 | — | |
| S15 | 33-34 | — | |
| 1 | 35-36 | | |
| 2 | 37-38 | | |
| 3 | 39-40 | | |
| 4 | 41-42 | | |
| 5 | 43-44 | | |
| 6 | 45-46 | | |
| 7 | 47-48 | | |
| Control | 49-50 | | — |
| 8 | 51-52 | | |
| 9 | 53-54 | | |
| 10 | 55-56 | | |
| 11 | 57-58 | | |
| 12 | 59-60 | | |
| 13 | 61-62 | | |
| 14 | 63-64 | | |
| 15 | 65-66 | | |
| 16 | 67-68 | | |
| 17 | 69-70 | | |
| Control | 71-72 | | — |
| 18 | 73-74 | | |
| 19 | 75-76 | | |
| 20 | 77-78 | | |
| 21 | 79-80 | | |
| 22 | 81-82 | | |
| 23 | 83-84 | | |
| 24 | 85-86 | | |
| 25 | 87-88 | | |
| 26 | 89-90 | | |
| 27 | 91-92 | | |
| 28 | 93-94 | | |
| Blank | 95-96 | | — |

TABLE 7
Content of the different microtubes

| Tube # | Sample | Content |
|---|---|---|
| 1 | Blank | Buffer B |
| 2 | Control | Buffer B |
| 3 | S1 | AAMU or 1X |
| 4 | S2 | AAMU or 1X |
| 5 | S3 | AAMU or 1X |
| 6 | S4 | AAMU or 1X |
| 7 | S5 | AAMU or 1X |
| 8 | S6 | AAMU or 1X |
| 9 | S7 | AAMU or 1X |
| 10 | S8 | AAMU or 1X |
| 11 | S9 | AAMU or 1X |
| 12 | S10 | AAMU or 1X |
| 13 | S11 | AAMU or 1X |
| 14 | S12 | AAMU or 1X |
| 15 | S13 | AAMU or 1X |
| 16 | S14 | AAMU or 1X |
| 17 | S15 | AAMU or 1X |
| 18 | 1 | Dil. Urine |
| 19 | 2 | Dil. Urine |
| 20 | 3 | Dil. Urine |
| 21 | 4 | Dil. Urine |
| 22 | 5 | Dil. Urine |
| 23 | 6 | Dil. Urine |
| 24 | Control | Buffer B |
| 25 | 7 | Dil. Urine |
| 26 | 8 | Dil. Urine |
| 27 | 9 | Dil. Urine |
| 28 | 10 | Dil. Urine |
| 29 | 11 | Dil. Urine |
| 30 | 12 | Dil. Urine |
| 31 | 13 | Dil. Urine |
| 32 | 14 | Dil. Urine |
| 33 | 15 | Dil. Urine |
| 34 | 16 | Dil. Urine |
| 35 | 17 | Dil. Urine |
| 36 | Control | Buffer B |
| 37 | 18 | Dil. Urine |
| 38 | 19 | Dil. Urine |
| 39 | 20 | Dil. Urine |
| 40 | 21 | Dil. Urine |
| 41 | 22 | Dil. Urine |
| 42 | 23 | Dil. Urine |
| 43 | 24 | Dil. Urine |
| 44 | 25 | Dil. Urine |
| 45 | 26 | Dil. Urine |
| 46 | 27 | Dil. Urine |
| 47 | 28 | Dil. Urine |
| 48 | Blank | Buffer B |

Solutions

Buffer A: Dissolve the powder of one vial A/50 mL water.

Buffer B: Dissolve the content of one vial B/100 mL water.

Buffer C: Dissolve the content of one vial C/50 mL water. Add 25 mL of Tween™ 20.

Buffer D: Dissolve the content of one vial D /25 mL water. Add 25 mL of Tween™ 20.

0.05% Tween™ 20: Add 25 μL of Tween™ 20 to a 100-mL erlenmeyer flask containing 50 mL of water.

2.5 N HCl: 41.75 mL of 12 N HCl/200 mL water. Store in a 250-mL glass bottle.

AAMU-HRP conjugate: Add 9 mL of Buffer C to a 15-mL glass test tube. Add 90 μL of AAMU-HRP stock solution.

1X-HRP conjugate: Add 9 mL of 2% BSA solution to a 15-mL glass test tube. Add 90 μL 1X-HRP stock solution.

Buffer E-H₂O₂: Dissolve the content of one vial E-substrate/50 ml water. Add 25 μL of a 30% H₂O₂ solution (prepared just prior to adding to the microtiter plate wells).

TABLE 8
Standard solutions of AAMU and 1X
(diluted with buffer B)

| | AAMU | | 1X |
|---|---|---|---|
| Standard | [AAMU] | Standard | [1X] |
| 1 | 1.12 × 10⁻⁴ M | 1 | 2.00 × 10⁻⁴ M |
| 2 | 6.00 × 10⁻⁵ M | 2 | 1.12 × 10⁻⁴ M |
| 3 | 3.56 × 10⁻⁵ M | 3 | 6.00 × 10⁻⁵ M |
| 4 | 2.00 × 10⁻⁵ M | 4 | 3.56 × 10⁻⁵ M |
| 5 | 6.00 × 10⁻⁶ M | 5 | 2.00 × 10⁻⁵ M |
| 6 | 3.56 × 10⁻⁶ M | 6 | 1.12 × 10⁻⁵ M |
| 7 | 2.00 × 10⁻⁶ M | 7 | 6.00 × 10⁻⁶ M |
| 8 | 1.12 × 10⁻⁶ M | 8 | 3.56 × 10⁻⁶ M |
| 9 | 6.00 × 10⁻⁷ M | 9 | 2.00 × 10⁻⁶ M |
| 10 | 3.56 × 10⁻⁷ M | 10 | 1.12 × 10⁻⁶ M |
| 11 | 2.00 × 10⁻⁷ M | 11 | 6.00 × 10⁻⁷ M |
| 12 | 1.12 × 10⁻⁷ M | 12 | 3.56 × 10⁻⁷ M |
| 13 | 6.00 × 10⁻⁸ M | 13 | 2.00 × 10⁻⁷ M |
| 14 | 3.56 × 10⁻⁸ M | 14 | 1.12 × 10⁻⁷ M |
| 15 | 2.00 × 10⁻⁸ M | 15 | 6.00 × 10⁻⁸ M |

Conditions of the ELISA

Add 50 μL/well of AAMU-HRP (or 1X-HRP) conjugate solution, starting from the last row. Add 50 μL/well of diluted urine samples in duplicate, standards (see Table 8), blank with a micropipet (0-200 μL), starting from well # 96 (see Table 6). Cover the plate and mix gently by vortexing for several seconds. Leave the plate at room temperature for 3 h. Wash 3 times with 100 μL/well with buffer C, using a microtiter plate washer. Wash 3 times with 100 μL/well with the 0.05% Tween™ 20 solution. Add 150 μL/well of Buffer E-H₂O₂ (prepared just prior adding to the microtiter plate wells). Shake 20-30 min at room temperature with an orbital shaker. Add 50 μL/well of a 2.5 N HCl solution. Shake 3 min with the orbital shaker at room temperature. Read the absorbance of the wells with microtiter plate reader at 490 nm. Print the sheet of data and properly identify the data sheet.

Calculation of the [AAMU] and [1X] in Urine Samples From the Data

Draw a Table 9 with a computer. Using the data sheet of the microtiter plate reader, enter the average absorbance values of blanks, controls (no free hapten present), standards and samples in Table 9. Draw the calibration curve on a semi-logarithmic plot (absorbance at 490 nm as a function of the standard concentrations) using sigma plot (or other plot software). Find the [AAMU] (or [1X]) in the microtiter well of the unknown from the calibration curve and enter the data in Table 10. Multiply the [AAMU] (or [1X]) of the unknown by the dilution factor and enter the result in the corresponding case of Table 10.

The compositions of the buffers used in the ELISA kit are shown in Table 11.

TABLE 9
Average absorbance values of
samples in the microtiter plate

| Sample | Well # | A₄₉₀ |
|---|---|---|
| Blank | 1-2 | |
| Control | 3-4 | |
| S1 | 5-6 | |
| S2 | 7-8 | |
| S3 | 9-10 | |
| S4 | 11-12 | |
| S5 | 13-14 | |
| S6 | 15-16 | |
| S7 | 17-18 | |
| S8 | 19-20 | |
| S9 | 21-22 | |
| S10 | 23-24 | |
| S11 | 25-26 | |
| S12 | 27-28 | |
| S13 | 29-30 | |
| S14 | 31-32 | |
| S15 | 33-34 | |
| 1 | 35-36 | |
| 2 | 37-38 | |
| 3 | 39-40 | |
| 4 | 41-42 | |
| 5 | 43-44 | |
| 6 | 45-46 | |
| 7 | 47-48 | |
| Control | 49-50 | |
| 8 | 51-52 | |
| 9 | 53-54 | |
| 10 | 55-56 | |
| 11 | 57-58 | |
| 12 | 59-60 | |
| 13 | 61-62 | |
| 14 | 63-64 | |
| 15 | 65-66 | |
| 16 | 67-68 | |
| 17 | 69-70 | |
| Control | 71-72 | |
| 18 | 73-74 | |
| 19 | 75-76 | |
| 20 | 77-78 | |
| 21 | 79-80 | |
| 22 | 81-82 | |
| 23 | 83-84 | |
| 24 | 85-86 | |
| 25 | 87-88 | |
| 26 | 89-90 | |
| 27 | 91-92 | |
| 28 | 93-94 | |
| Blank | 95-96 | |

TABLE 10
AAMU (or 1X) concentrations in urine samples

| Sample | D.F. | [AAMU] | [AAMU] × D.F. |
|---|---|---|---|
| 1 | | | |
| 2 | | | |
| 3 | | | |
| 4 | | | |
| 5 | | | |
| 6 | | | |
| 7 | | | |
| 8 | | | |
| 9 | | | |
| 10 | | | |
| 11 | | | |
| 12 | | | |
| 13 | | | |
| 14 | | | |
| 15 | | | |
| 16 | | | |
| 17 | | | |
| 18 | | | |
| 19 | | | |
| 20 | | | |
| 21 | | | |
| 22 | | | |
| 23 | | | |
| 24 | | | |
| 25 | | | |
| 26 | | | |
| 27 | | | |
| 28 | | | |
| 29 | | | |

TABLE 11
Composition of the different buffer

| Buff- | | | Concen. | [P] |
|---|---|---|---|---|
| er | pH | Composition | (mM) | (mM) |
| A | 7.50 | 0.15629 g/100 mL NaH₂PO₄ | 11.325 | |
| | | 1.622 g/100 mL Na₂HPO₄.7 H₂O | 60.099 | |
| | | 1.778 g/100 mL (total weight) | | 71.424 |
| B | 7.50 | 0.1210191 g/100mL Na H₂PO₄ | 8.769 | |
| | | 1.11309 g /100 mL of Na₂HPO₄.7H₂O | 41.23 | |
| | | 1.2341 g/100 mL (total weight) | | 49.999 |
| C | 7.50 | 1 g/100 mL of BSA | — | |
| | | 0.1210191 g/100 mL of NaH₂PO₄ | 8.769 | |
| | | 1.11309 g/100 mL of Na₂HPO₄.7H₂O | 41.23 | |
| | | 2.2341 g/100 mL (total weight) | | 49.999 |
| D | 7.50 | 2 g/100 mL of BSA | | |
| | | 0.1210191 g/100 mL of NaH₂PO₄ | 8.769 | |
| | | 1.11309 g/100 mL of Na₂HPO₄.7H₂O | 41.23 | |
| | | 3.2341 g/100 mL (total weight) | | 49.999 |
| E | 5.00 | 0.52508 g/100 mL of citric acid | 25 | |
| | | 1.34848 g/100 mL of Na₂HPO₄.7H₂O | 50 | |
| | | 40 mg/100 mL of o-phenylenediamine | | |
| | | hydrochloride | | |
| | | 1.913567 g/100 mL (total weight) | | — |

### EXAMPLE II

### A Competitive Antigen ELISA for CYP2C19 Phenotyping

Buffers and water without additives are filtered through 0.45 μM millipore filters and kept for one week, except the substrate buffer which was freshly prepared. BSA, antibodies, Tween™ 20 and horse radish peroxidase are added to buffers and water just prior to use.

Preparation of Urine Samples

Urine samples are usually collected four hours after ingestion of 100 mg of racemic mephenytoin and stored at −20° C. as 1-mL aliquots in 1.5 mL microtubes. For the ELISA, the urine samples are diluted with isotonic sodium phosphate buffer, pH 7.5 (310 mosM) to give concentrations of S-mephenytoin or R-mephenytoin no higher than 3×10⁻⁶ M in the microtiter plate wells. Just prior to the ELISA, samples are mixed in a 1:1 ratio (e.g. 100 μl:100 μl) with either the R-mephenytoin-HRP or the S-mephenytoin-HRP conjugate(12 mg ml⁻¹).

Standard Solutions of R-Mephenytoin or S-Mephenytoin for ELISA

Prepare a 100 mL stock solution of S-mephenytoin or R-mephenytoin at concentrations of 6.00×10⁻⁴ M in the 310 mosM sodium phosphate buffer, pH 7.5 (IPB) in a 100 mL volumetric flask. Stir the solution to insure complete solubilization.

Store the stock solutions as 1 mL aliquots at −20° C. On the day of the ELISA, thaw one aliquot and warm up at room temperature. Prepare the following standard solutions of the above compounds:

| Standard # | [Compound] | | Composition |
|---|---|---|---|
| 1 | 6.00 × 10⁻⁴ | M | Stock Solution |
| 2 | 2.00 × 10⁻⁴ | M | 200 μL S1 + 400 μL IPB |
| 3 | 1.12 × 10⁻⁴ | M | 200 μL S1 + 868 μL IPB |
| 4 | 6.00 × 10⁻⁵ | M | 100 μL S1 + 900 μL IPB |
| 5 | 3.56 × 10⁻⁵ | M | 60 μL S1 + 951 μL IPB |
| 6 | 2.00 × 10⁻⁵ | M | 100 μL S2 + 900 μL IPB |
| 7 | 1.12 × 10⁻⁵ | M | 100 μL S3 + 900 μL IPB |
| 8 | 6.00 × 10⁻⁶ | M | 100 μL S4 + 900 μL IPB |
| 9 | 3.56 × 10⁻⁶ | M | 100 μL S5 + 900 μL IPB |
| 10 | 2.00 × 10⁻⁶ | M | 100 μL S6 + 900 μL IPB |
| 11 | 1.12 × 10⁻⁶ | M | 100 μL S7 + 900 μL IPB |
| 12 | 6.00 × 10⁻⁷ | M | 100 μL S8 + 900 μL IPB |
| 13 | 3.56 × 10⁻⁷ | M | 100 μL S9 + 900 μL IPB |
| 14 | 2.00 × 10⁻⁷ | M | 100 μL S10 + 900 μL IPB |
| 15 | 1.12 × 10⁻⁷ | M | 100 μL S11 + 900 μL IPB |
| 16 | 6.00 × 10⁻⁸ | M | 100 μL S12 + 900 μL IPB |
| 17 | 3.56 × 10⁻⁸ | M | 100 μL S13 + 900 μL IPB |
| 18 | 2.00 × 10⁻⁸ | M | 100 μL S14 + 900 μL IPB |
| 19 | 2.00 × 10⁻⁹ | M | 100 μL S15 + 900 μL IPB |
| 20 | 2.00 × 10⁻¹⁰ | M | 100 μL S15 + 900 μL IPB |
| 21 | 2.00 × 10⁻¹¹ | M | 100 μL S15 + 900 μL IPB |
| 22 | 2.00 × 10⁻¹² | M | 100 μL S15 + 900 μL IPB |
| 23 | 2.00 × 10⁻¹³ | M | 100 μL S15 + 900 μL IPB |

ELISA Conditions

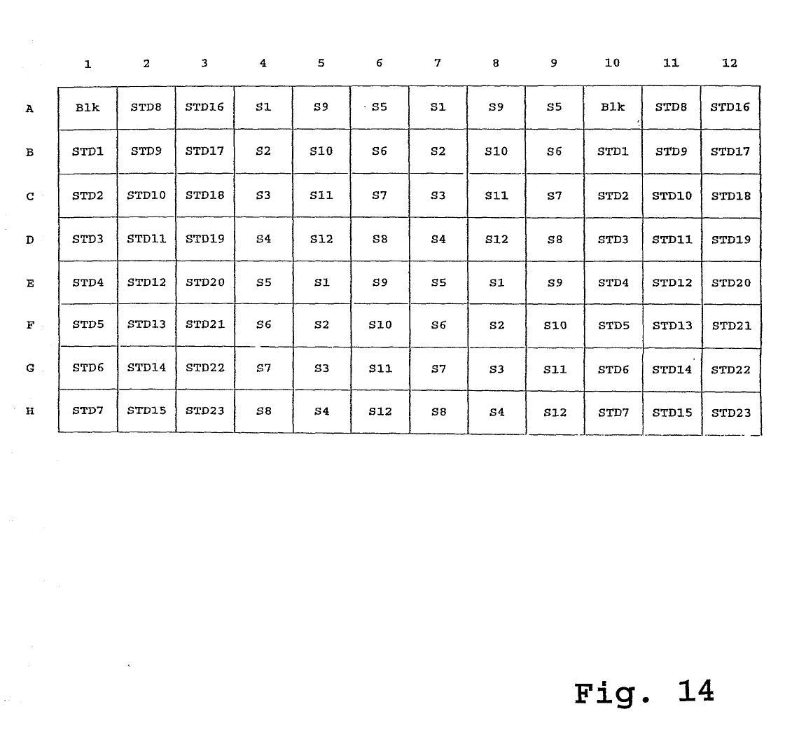

Wells of the ELISA plate are washed with a Nunc-Immuno wash 12 washer. Sixteen milliliters (16 mL) of a solution of 6.6 μg ml⁻¹ of isolated IgG antibodies is prepared in a 100 mM sodium carbonate buffer, pH 9.6, and 150 μL of this solution is pipetted in each well of a microtiter plate using a eight channel pipet (Brinkmann Transferpette™-8 50-200 μL) and 200 μL Flex tips from Brinkmann). After coating the wells with antibodies at 4° C. for 20 h, the wells are washed 3 times with the isotonic sodium phosphate buffer containing 0.05% Tween™ 20 (IPBT) and properly drained by inverting the plate and absorbing the liquid on piece of paper towel. Thirty milliliters (30 mL) of a solution of a IPBT solution containing 1% BSA is prepared and 150 μL of this solution is pipetted in each well using a eight channel pipet (Brinkmann Transferpette™-8 50-200 μL) and 200 μL yellow tips (Sarstedt yellow tips for P200 Gilson Pipetman). After 3 h at room temperature, the wells were washed 3 times with IPBT solution and drained. Four hundred microliters (400 μl) of sample or standard for determination of R-mephenytoin or S-mephenytoin are prepared (as described in previous sections) in 1.5 mL microtubes using Sarstedt yellow tips and a P200 Gilson Pipetman. Two hundred microliters (200 μL) of each sample/standard are pipetted in duplicate in a Falcon 96 well microtest tissue culture plate according to the pattern shown in FIG. 14, using Sarstedt yellow tips and a P200 Gilson Pipetman. Using an eight channel pipet (Brinkmann Transferpette™-8 50-200 μL) and changing the tips of the eight channel pipet (200 μL Flex tips from Brinkmann) at each row, 150 μL of sample/standard are transferred in the corresponding wells of a 96 well ELISA microtiter plate coated with antibodies. After the addition of the samples and standards, the microtiter plates are covered and left standing at room temperature for 2 h. While the plate is left standing the substrate buffer without the hydrogen peroxide and o-phenylenediamine hydrochloride is prepared (25 mM citric acid and 50 mM sodium phosphate dibasic buffer, pH 5.0). The microtiter plate is washed 3 times with the IPBT solution and 3 times with a 0.05% Tween™ solution and drained. 50 μL of hydrogen peroxide and 40 mg of o-phenylenediamine are added to the substrate buffer. One hundred and fifty microliters (150 μL) of the substrate buffer solution is then added to each wells using a eight channel pipet (Brinkmann Transferpette™-8 50-200 μL) and 200 μL Flex tips (Brinkmann). The microtiter plate is covered and shaken for 25-30 min at room temperature and the enzymatic reaction is stopped by adding 50 μL/well of a 2.5 M HCl solution using an eight channel pipet (Brinkmann Transferpette™-8 50-200 μL) and 200 μL Flex tips (Brinkmann). After gently shaking for 3 min, the absorbance is read at 490 nm with a microplate reader.

Determination of S-Mephenytoin and R-Mephenytoin in Urine Samples With the ELISA Kit

The contents of an ELISA kit for determining CYP2C19 phenotype are exemplified in Table 12.

TABLE 12
Content of the ELISA kit and Conditions of Storage

| | | | | | Storage |
|---|---|---|---|---|---|
| Item | Unit | State | Amt. | | Conditions |
| Tween 20 ™ 20 | 1 vial | liquid | 250 | μL/vial | 4° C. |
| H₂O₂ | 1 vial | liquid | 250 | μL/vial | 4° C. |
| S-Mephenytoin-HRP | 1 vial | liquid | 250 | μL/vial | 4° C. |
| R-Mephenytoin-HRP | 1 vial | liquid | 250 | μL/vial | 4° C. |
| Buffer* A | 4 vials | Solid | 0.8894 | g/vial | 4° C. |
| Buffer* B | 6 vials | Solid | 1.234 | g/vial | 4° C. |
| Buffer* C | 6 vials | Solid | 1.1170 | g/vial | 4° C. |
| Buffer* D | 6 vials | Solid | 0.8082 | g/vial | 4° C. |
| Plate (5- | 2 | Solid | | — | 4° C. |
| Mephenytoin-Ab) | | | | | |
| Plate (R- | 2 | Solid | | — | 4° C. |
| Mephenytoin-Ab) | | | | | |
| Buffer* E | 6 vials | Solid | 0.9567 | g/vial | −20° C. |
| Standards (5- | 14 | Liquid | 200 | μL | −20° C. |
| Mephenytoin) | vials | | | | |
| Standards (R- | 14 | Liquid | 200 | μL | −20° C. |
| Mephenytoin) | vials | | | | |
| 1N NaOH | 1 | Liquid | 15 | mL | 20° C. |
| | | bottle | | | |
| 1N HCl | 1 | Liquid | 15 | mL | 20° C. |
| | | bottle | | | |

| | |
|---|---|
| SOLUTIONS | |
| Buffer A: | Dissolve the content of 1 vial B/25 mL. |
| Buffer B: | Dissolve the content of 1 vial B/100 mL. |
| Buffer C: | Dissolve the content of one vial C/50 mL. |
| | Pipet 25 mL of Tween ™ 20. |
| Buffer D: | Dissolve the content of one vial D/25 mL. |
| | Pipet 25 mL of Tween ™ 20. |
| 0.05% Tween ™ 20: | Pipet 25 mL of Tween ™ 20 in a 100 mL |
| | erlenmeyer flask containing 50 mL of |
| | water. |
| 2.5N HCl: | 41.75 mL of 12 N HCl/200 mL. Store in |
| | a 250 mL glass bottle. |
| S-mephenytoin-HRP | Pipet 9 mL of Buffer C in a |
| conjugate: | 15 mL glass test tube. |
| | Pipet 90 μL of |
| | S-mephenytoin-HRP stock |
| | solution. |
| R-mephenytoin-HRP | Pipet 9 mL of Buffer C in a |
| conjugate: | 15 mL glass test tube. |
| | Pipet 90 μL of |
| | R-mephenytoin-HRP stock |
| | solution. |
| Buffer E - H₂O₂: | Dissolve the contents of 1 vial |
| | E-substrate/50 mL water. Pipet 25 μL |
| | of a 30% H₂O₂ solution (prepared |
| | fresh). |

*Composition of all buffers is described in Table 11

Dilutions of Urine Samples for the Determinations of [S-Mephenytoin] and [S-Mephenytoin] by ELISA

The dilutions of urine samples required for determinations of S-Mephenytoin and R-Mephenytoin are a function of the sensitivity of the competitive antigen ELISA and of S-Mephenytoin and R-Mephenytoin concentrations in urine samples. It is suggested to dilute the urine samples by a factor so S-Mephenytoin and R-Mephenytoin are about 3×10⁻⁶ M in the well of the microtiter plate (see table 13).

TABLE 13
Dilution Factors for Identifying S-Mephenytoin
and R-Mephenytoin Concentrations

| | Microtube # | | | | | | | |
|---|---|---|---|---|---|---|---|---|
| Dilution Factor | 20x | 40x | 50x | 80x | 100x | 150x | 200x | 400x |
| Solution | 1 | 2 | 3 | 4 | 5 | 6 | 7 | 8 |
| Urine | 500 | 250 | 200 | 125 | 100 | 66.7 | 50 | 25 |
| Sample (μL)^{a} | | | | | | | | |
| 10 x diluted | | | | | | | | |
| Buffer B (μL) | 500 | 750 | 800 | 875 | 900 | 933.3 | 950 | 975 |

^{a}Vortex the microtubes containing the urine sample before pipetting.
Store the diluted urine samples are stored at −20° C. in a box for microtubes.

Determination of [S-Mephenytoin] and [R-Mephenytoin] in Diluted Urine Samples by ELISA

Precautions

The HRP substrate (p-nitrophenolphosphate) is carcinogenic. Wear surgical gloves when handling Buffer E (substrate buffer). Each sample is determined in duplicate. An excellent pipetting technique is required. When this technique is mastered the absorbency values of duplicates should be within less than 5%. Buffers C, D, E are freshly prepared. Buffer E-H₂O₂ is prepared just prior to pipetting in the microtiter plate wells.

Preparation of Samples

Prepare Table 14 with a computer and print it. This table shows the contents of each well of a 96 well microtiter plate. Enter the name of the urine sample (or number) at the corresponding well positions in Table 14. Select the dilution factor (D.F.) of each urine sample and enter at the corresponding position in Table 14. Enter the dilution of each urine sample with buffer B at the corresponding position in Table 14: for example, a D.F. of 100 (100 μL of 10× diluted urine sample +900 μL buffer B), enter 100/900. See “Dilutions of Urine Samples . . . ” procedure described above for the preparation of the different dilutions. Prepare the different dilutions of the urine samples in 1.5 mL microtubes using a styrofoam support for 100 microtubes. Standard solutions of concentrations indicated in Table 15 are preferably provided with the kit of the present invention. Prepare Table 16 with a computer and print it. Using a styrofoam port (100 microtubes), prepare the following microtubes in the order indicated in Table 16.

TABLE 14
Positions of Blanks, Control and
Urine Samples in a Microtiter plate

| Sample | Well # | D.F. | Dil. |
|---|---|---|---|
| Blank | 1-2 | — | |
| Control | 3-4 | — | |
| S1 | 5-6 | — | |
| S2 | 7-8 | — | |
| S3 | 9-10 | — | |
| S4 | 11-12 | — | |
| S5 | 13-14 | — | |
| S6 | 15-16 | — | |
| S7 | 17-18 | — | |
| S8 | 19-20 | — | |
| S9 | 21-22 | — | |
| S10 | 23-24 | — | |
| S11 | 25-26 | — | |
| S12 | 27-28 | — | |
| S13 | 29-30 | — | |
| S14 | 31-32 | — | |
| S15 | 33-34 | — | |
| 1 | 35-36 | | |
| 2 | 37-38 | | |
| 3 | 39-40 | | |
| 4 | 41-42 | | |
| 5 | 43-44 | | |
| 6 | 45-46 | | |
| 7 | 47-48 | | |
| Control | 49-50 | | — |
| 8 | 51-52 | | |
| 9 | 53-54 | | |
| 10 | 55-56 | | |
| 11 | 57-58 | | |
| 12 | 59-60 | | |
| 13 | 61-62 | | |
| 14 | 63-64 | | |
| 15 | 65-66 | | |
| 16 | 67-68 | | |
| 17 | 69-70 | | |
| Control | 71-72 | | — |
| 18 | 73-74 | | |
| 19 | 75-76 | | |
| 20 | 77-78 | | |
| 21 | 79-80 | | |
| 22 | 81-82 | | |
| 23 | 83-84 | | |
| 24 | 85-86 | | |
| 25 | 87-88 | | |
| 26 | 89-90 | | |
| 27 | 91-92 | | |
| 28 | 93-94 | | |
| Blank | 95-96 | | — |

TABLE 15
Standard Solutions of S-mephenytoin and R-mephenytoin
(Diluted with Buffer B)

| Standard | S-mephenytoin | Standard | R-mephenytoin |
|---|---|---|---|
| 1 | 1.12 × 10⁻⁴ M | 1 | 1.12 × 10⁻⁴ M |
| 2 | 6.00 × 10⁻⁵ M | 2 | 6.00 × 10⁻⁵ M |
| 3 | 3.56 × 10⁻⁵ M | 3 | 3.56 × 10⁻⁵ M |
| 4 | 2.00 × 10⁻⁵ M | 4 | 2.00 × 10⁻⁵ M |
| 5 | 6.00 × 10⁻⁶ M | 5 | 6.00 × 10⁻⁶ M |
| 6 | 3.56 × 10⁻⁶ M | 6 | 3.56 × 10⁻⁶ M |
| 7 | 2.00 × 10⁻⁶ M | 7 | 2.00 × 10⁻⁶ M |
| 8 | 1.12 × 10⁻⁶ M | 8 | 1.12 × 10⁻⁶ M |
| 9 | 6.00 × 10⁻⁷ M | 9 | 6.00 × 10⁻⁷ M |
| 10 | 3.56 × 10⁻⁷ M | 10 | 3.56 × 10⁻⁷ M |
| 11 | 2.00 × 10⁻⁷ M | 11 | 2.00 × 10⁻⁷ M |
| 12 | 1.12 × 10⁻⁷ M | 12 | 1.12 × 10⁻⁷ M |
| 13 | 6.00 × 10⁻⁸ M | 13 | 6.00 × 10⁻⁸ M |
| 14 | 3.56 × 10⁻⁸ M | 14 | 3.56 × 10⁻⁸ M |
| 15 | 2.00 × 10⁻⁸ M | 15 | 2.00 × 10⁻⁸ M |

TABLE 16
Content of Microtubes for
CYP2C19 phenotyping ELISA

| Tube # | Sample | Content |
|---|---|---|
| 1 | Blank | Buffer B |
| 2 | Control | Buffer B |
| 3 | S1 | S-Mephenytoin/R-Mephenytoin |
| 4 | S2 | S-Mephenytoin/R-Mephenytoin |
| 5 | S3 | S-Mephenytoin/R-Mephenytoin |
| 6 | S4 | S-Mephenytoin/R-Mephenytoin |
| 7 | S5 | S-Mephenytoin/R-Mephenytoin |
| 8 | S6 | S-Mephenytoin/R-Mephenytoin |
| 9 | S7 | S-Mephenytoin/R-Mephenytoin |
| 10 | S8 | S-Mephenytoin/R-Mephenytoin |
| 11 | S9 | S-Mephenytoin/R-Mephenytoin |
| 12 | S10 | S-Mephenytoin/R-Mephenytoin |
| 13 | S11 | S-Mephenytoin/R-Mephenytoin |
| 14 | S12 | S-Mephenytoin/R-Mephenytoin |
| 15 | S13 | S-Mephenytoin/R-Mephenytoin |
| 16 | S14 | S-Mephenytoin/R-Mephenytoin |
| 17 | S15 | S-Mephenytoin/R-Mephenytoin |
| 18 | 1 | Dil. Urine |
| 19 | 2 | Dil. Urine |
| 20 | 3 | Dil. Urine |
| 21 | 4 | Dil. Urine |
| 22 | 5 | Dil. Urine |
| 23 | 6 | Dil. Urine |
| 24 | Control | Buffer B |
| 25 | 7 | Dil. Urine |
| 26 | 8 | Dil. Urine |
| 27 | 9 | Dil. Urine |
| 28 | 10 | Dil. Urine |
| 29 | 11 | Dil. Urine |
| 30 | 12 | Dil. Urine |
| 31 | 13 | Dil. Urine |
| 32 | 14 | Dil. Urine |
| 33 | 15 | Dil. Urine |
| 34 | 16 | Dil. Urine |
| 35 | 17 | Dil. Urine |
| 36 | Control | Buffer B |
| 37 | 18 | Dil. Urine |
| 38 | 19 | Dil. Urine |
| 39 | 20 | Dil. Urine |
| 40 | 21 | Dil. Urine |
| 41 | 22 | Dil. Urine |
| 42 | 23 | Dil. Urine |
| 43 | 24 | Dil. Urine |
| 44 | 25 | Dil. Urine |
| 45 | 26 | Dil. Urine |
| 46 | 27 | Dil. Urine |
| 47 | 28 | Dil. Urine |
| 48 | Blank | Buffer B |

Conditions of the ELISA

Pipet 50 μL/well of S-mephenytoin-HRP (R-mephenytoin) conjugate solution starting from the last row. Pipet 50 μL/well of diluted urine samples in duplicate, standards, blank with a micropipet (0-200 μL), starting from well # 96 (see Table 17). Cover the plate and mix gently by vortexing for several seconds. Leave the plate at room temperature for 3 h. Wash 3 times with 100 μL/well Buffer C, using a microtiter plate washer. Wash 3 times with 100 μL/well 0.05% Tween™-20 solution. Pipet 150 μL/well of Buffer E- H₂O₂ (prepared just prior to pipetting in the microtiter plate wells). Shake for 20-30 min at room temperature using an orbital shaker. Pipet 50 μL/well of a 2.5N HCl solution. Shake 3 min with the orbital shaker at room temperature. Read the absorbance of the wells with a microtiter plate reader at 490 nm. Print the sheet of data and properly label.

Calculation of the [S-Mephenytoin] and [R-Mephenytoin] in Urine Samples From the Data

Draw Table 17 with a computer. Using the data sheet of the microtiter plate reader, enter the average absorbance values of blanks, controls (no free hapten present), standards and samples in Table 17. Draw the calibration curve on a semi-logarithmic plot (absorbance at 490 nm as a function of the standard concentrations) using sigma-plot (or other plot software). Find the [S-mephenytoin] (or [R-mephenytoin]) in the microtiter well of the unknowns from the calibration curve and enter the data in Table 18. Multiply the [S-mephenytoin] (or [R-mephenytoin]) of the unknown by the dilution factor and enter the result in the corresponding cell of Table 18.

The compositions of the buffers used in the ELISA kit are shown in Table 19.

TABLE 17
Average Absorbance Values of
Samples in the Microtiter plate

| Sample | Well # | A₄₉₀ |
|---|---|---|
| Blank | 1-2 | |
| Control | 3-4 | |
| S1 | 5-6 | |
| S2 | 7-8 | |
| S3 | 9-10 | |
| S4 | 11-12 | |
| S5 | 13-14 | |
| S6 | 15-16 | |
| S7 | 17-18 | |
| S8 | 19-20 | |
| S9 | 21-22 | |
| S10 | 23-24 | |
| S11 | 25-26 | |
| S12 | 27-28 | |
| S13 | 29-30 | |
| S14 | 31-32 | |
| S15 | 33-34 | |
| 1 | 35-36 | |
| 2 | 37-38 | |
| 3 | 39-40 | |
| 4 | 41-42 | |
| 5 | 43-44 | |
| 6 | 45-46 | |
| 7 | 47-48 | |
| Control | 49-50 | |
| 8 | 51-52 | |
| 9 | 53-54 | |
| 10 | 55-56 | |
| 11 | 57-58 | |
| 12 | 59-60 | |
| 13 | 61-62 | |
| 14 | 63-64 | |
| 15 | 65-66 | |
| 16 | 67-68 | |
| 17 | 69-70 | |
| Control | 71-72 | |
| 18 | 73-74 | |
| 19 | 75-76 | |
| 20 | 77-78 | |
| 21 | 79-80 | |
| 22 | 81-82 | |
| 23 | 83-84 | |
| 24 | 85-86 | |
| 25 | 87-88 | |
| 26 | 89-90 | |
| 27 | 91-92 | |
| 28 | 93-94 | |
| Blank | 95-96 | |

TABLE 18
S-mephenytoin and R-mephenytoin Concentrations in Urine Samples

| | | | |
|---|---|---|---|
| Sample | D.F. | [mephenytoin] | [mephenytoin] × D.F. |

1
2
3
4
5
6
7
8
9
10
11
12
13
14
15
16
17
18
19
20
21
22
23
24
25
26
27
28
29

TABLE 19
Composition of the Different Buffers

| | | | Conc. | [P] |
|---|---|---|---|---|
| Buffer | pH | Composition | (mM) | (mM) |
| A | 7.50 | 0.15629 g/100 mL NaH₂PO₄ | 11.325 | 71.424 |
| | | 1.622 g/100 mL Na₂HPO₄.7H₂O | 60.099 | |
| | | 1.778 g/100 mL (total weight) | | |
| B | 7.50 | 0.1210191 g/100 mL NaH₂PO₄ | 8.769 | 49.999 |
| | | 1.11309 g/100 mL Na₂HPO₄.7H₂O | 41.23 | |
| | | 1.2341 g/100 mL (total weight) | | |
| C | 7.50 | 1 g/100 mL BSA | 8.769 | 49.999 |
| | | 0.1210191 g/100 mL NaH₂PO₄ | 41.23 | |
| | | 1.11309 g/100 mL Na₂HPO₄.7H₂O | | |
| | | 2.2341 g/100 mL (total weight) | | |
| D | 7.50 | 2 g/100 mL BSA | 8.769 | 49.999 |
| | | 0.1210191 g/100 mL NaH₂PO₄ | 41.23 | |
| | | 1.11309 g/100 mL Na₂HPO₄.7H₂O | | |
| | | 3.2341 g/100 mL (total weight) | | |
| E | 5.00 | 0.52508 g/100 mL of citric acid | 25 | — |
| | | 1.34848 g/100 mL Na₂HPO₄.7H₂O | 50 | |
| | | 40 mg/100 mL of o-phenylenedi- | | |
| | | amine hydrochloride | | |
| | | 1.913567 g/100 mL (total weight) | | |

Discussion

In the form of a kit, the present invention provides a convenient and effective tool for use in both a clinical and laboratory environment. The kit of the present invention is particularly suited for use by a physician or other qualified personnel in a clinic, whereby a fast and accurate result can be easily obtained. According to an embodiment of the present invention, a ready-to-use kit is provided for fast and accurate determination of an individual's CYP2C19 phenotype. As a result of the convenience and ease of use of ELISA and/or kit of the present invention, a physician is provided with a tool for use in the individualization of treatment. A quick and accurate determination of an individual's CYP2C19 phenotype will allow a physician to consider this information before prescribing a treatment regime. In this manner, a method of individualizing treatment is also provided. In essence, a CYP2C19 phenotype characterization, according to the present invention can serve as a drug response profile specific to drugs known to be metabolized by CYP2C19 for the individual phenotyped. Furthermore, the ELISA and/or kit of the present invention may be used to screen individuals for their susceptibility to carcinogens or for their phenotypic compatibility with a particular drug known to metabolized completely or in part by CYP2C19.

The present invention provides a convenient and effective tool for use in both a clinical and laboratory environment. The present invention is particularly suited for use by a physician in a clinic, whereby phenotypic determinants of CYP2C19 can be quickly and easily obtained. According to an embodiment of the present invention, a ready-to-use kit is provided for fast and accurate determination of at least CYP2C19 determinants. The assay system and kit preferably employ antibodies specific to a plurality of substrates and/or forms thereof on a suitable substrate allowing for detection of the preferred substrates in a biological sample of an individual after consumption of a corresponding substrate (or probe drug). In accordance with a preferred embodiment of the present invention, the kit of the present invention will provide means to determine metabolic determinants for at least CYP2C19. The assay system of the present invention may be provided in a plurality of forms including but not limited to an ELISA assay, a high-throughput ELISA assay or a dipstick based ELISA assay.

The ELISA and/or kit of the present invention will employ antibodies specific to preferred metabolites, substrates and/or forms thereof known to acted on of the CYP2C19 metabolite pathway on a suitable substrate allowing for detection of the preferred metabolites in a biological sample of an individual after consumption of a corresponding probe substrate.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method of characterizing a CYP2C19-specific metabolic phenotype, wherein a plurality of phenotypic determinants are identified as corresponding to respective metabolic characteristics, said method comprising:
a) administering to an individual a probe substrate specific to the CYP2C19 metabolic pathway;
b) detecting metabolites of said CYP2C19 metabolic pathway in a biological sample from said individual in response to said probe substrate; and;
c) characterizing a phenotypic determinant of said CYP2C19 metabolic phenotype based on detected metabolites.

2. The method of claim 1 which further comprises a step i) after step b): i) quantifying a ratio of respective detected metabolites for said CYP2C19 metabolic pathway in said biological sample

3. The method of claim 2, wherein said ratio is selected from the group consisting of concentration ratio, molar ratio, chiral ratio, ratio of area under the curve and signal peak height ratio.

4. The method of claim 3 wherein said probe substrate is at least one probe substrate known to be metabolized by said CYP2C19 metabolic pathway.

5. The method of claim 4, wherein said probe substrate is other than an inducer or inhibitor of said metabolic pathway.

6. The method of claim 1, wherein said step b) and step c) is effected using an affinity complexation agent specific to each of said metabolites.

7. The method of claim 6, wherein said affinity complexation agent is an antibody.

8. The method of claim 7, wherein said antibody is a monoclonal antibody.

9. The method of claim 7, wherein said antibody is a polyclonal antibody.

10. The method of claim 6, wherein said affinity complexation agent is a molecular imprinted polymer.

11. The method of claim 6, wherein said affinity complexation agent is an aptmer.

12. The method of claim 6, wherein said affinity complexation agent is a receptor.

13. The method of claim 6, wherein said affinity complexation agent is an anticalin.

14. The method of claim 6, further comprising a ligand binding assay.

15. The method of claim 14, wherein said ligand binding assay is selected from the group consisting of immunoassay, enzyme-linked immunosorbent assay (ELISA), microarray formatted immunoassay and microarray formatted ELISA.

16. The method of claim 14, wherein said ligand binding assay is a rapid immunoassay (Dipstick assay).

17. The method of claim 16, wherein said rapid immunoassay is based on Rapid Analyte Measurement Platform (RAMP) technology.

18. The method of claim 16, wherein said rapid immunoassay is based on light-emitting immunoassay technology.

19. The method of claim 14, wherein said ligand binding assay is performed with a biosensor.

20. The method of claim 19, wherein said biosensor is an immunosensor.

21. The method of claim 19 wherein wherein the means of detection of said biosensor is an electrochemical sensor.

22. The method of claim 19, wherein the means of detection of said biosensor is an optical sensor.

23. The method of claim 19, wherein the means of detection of said biosensor is a microgravimetric sensor.

24. The method of claim 23, wherein said microgravimetric sensor is a quartz crystal microbalance (QCM).

25. The method of claim 1, wherein step b) is effected by using a qualitative detection instrument.

26. The method according to claim 1, wherein said phenotypic determinant of said CYP2C19 metabolic phenotype is an enzyme-specific determinant

27. The method of claim 2 wherein step a) is effected using a probe substrate specific to said CYP2C19 metabolic pathway.

28. The method of claim 27, wherein said CYP2C19 metabolic phenotype is characterized according to the method comprising:
a) administering to an individual a probe substrate specific to the CYP2C19 metabolic pathway;
b) detecting metabolites of said CYP2C19 metabolic pathway in a biological sample from said individual in response to said probe substrate; and;
c) characterizing a phenotypic determinant of said CYP2C19 metabolic phenotype based on detected metabolites.

29. The method claim 28 wherein said probe substrate is a racemic (50:50) mixture of mephenytoin.

30. The method of claim 29 wherein said forms of said substrate include R-mephenytoin and S-mephenytoin.

31. The method of claim 30 wherein said phenotypic determinant is characterized according to a chiral ratio of concentrations of said metabolites, as calculated by:
$$\frac{[ S \text{-} Mephenytoin ]}{[ R \text{-} Mephenytoin ]}$$

32. The method of claim 31 wherein when said phenotypic determinant is >0.8, said CYP 2C19 phenotype is characterized as a fast CYP2C19 metabolizer.

33. A method of using a CYP2C19 metabolic phenotype to select a drug treatment regimen for an individual, said method comprising, comparing a metabolic profile of a candidate drug with said CYP2C19 metabolic phenotype of said individual, and selecting said candidate drug for use in said treatment regimen for said individual when said CYP2C19 metabolic phenotypic is indicative of a phenotype having metabolic efficiency for said candidate drug.

34. The method of claim 31 wherein said multi-determinant metabolic phenotype is characterized according to the method comprising:
a) administering to an individual a probe substrate specific to the CYP2C19 metabolic pathway;
b) detecting metabolites of said CYP2C19 metabolic pathway in a biological sample from said individual in response to said probe substrate; and
c) characterizing a phenotypic determinant of said CYP2C19 metabolic phenotype based on detected metabolites.

35. A method of using a CYP2C19 metabolic phenotype to individualize a selected drug treatment regimen for an individual, wherein said CYP2C19 metabolic phenotype of said individual is determined; a safe and therapeutically effective dose of said drug treatment is determined for said individual based on said CYP2C19 phenotype; and said dose for use in said selected treatment regimen for said individual is selected based thereon.

36. The method of claim 35 wherein said multi-determinant metabolic phenotype is determined according to the method comprising:
a) administering to an individual a probe substrate specific to the CYP2C19 metabolic pathway;
b) detecting metabolites of said CYP2C19 metabolic pathway in a biological sample from said individual in response to said probe substrate; and
c) characterizing a phenotypic determinant of said CYP2C19 metabolic phenotype based on detected metabolites.

37. The method of claim 36, wherein said drug treatment is selected from a class or genus of compounds with similar metabolic profiles.

38. The method of claim 37 wherein said drug treatment regimen is selected according to the method comprising:
a) administering to an individual a probe substrate specific to the CYP2C19 metabolic pathway;
b) detecting metabolites of said CYP2C19 metabolic pathway in a biological sample from said individual in response to said probe substrate; and;
c) characterizing a phenotypic determinant of said CYP2C19 metabolic phenotype based on detected metabolites.

39. The method of claim 38, wherein said CYP2C19 metabolic phenotype is characterized according to the method comprising:
a) administering to an individual a probe substrate specific to the CYP2C19 metabolic pathway;
b) detecting metabolites of said CYP2C19 metabolic pathway in a biological sample from said individual in response to said probe substrate; and;
c) characterizing a phenotypic determinant of said CYP2C19 metabolic phenotype based on detected metabolites.

40. A method of treating an individual having a medical condition with a safe and therapeutically effective dose of a drug treatment known for use with said condition, said method comprising:
a) determining a CYP2C19 metabolic phenotype of said individual; and
b) administering a safe and therapeutically effective dose of at least one compound known for treating said condition,
wherein said at least one compound known for treating said condition has a metabolic profile corresponding to said individual's metabolic phenotype for said at least one compound as represented by said CYP2C19 metabolic phenotype.

41. The method of claim 40, wherein said multi-determinant metabolic phenotype is characterized according to the method comprising:
a) administering to an individual a probe substrate specific to the CYP2C19 metabolic pathway;
b) detecting metabolites of said CYP2C19 metabolic pathway in a biological sample from said individual in response to said probe substrate; and;
c) characterizing a phenotypic determinant of said CYP2C19 metabolic phenotype based on detected metabolites.

42. A method of selecting a treatment for an individual corresponding to said individual's CYP2C19 metabolic phenotype, said method comprising:
a) characterizing a CYP2C19 metabolic phenotype of said individual;
b) identifying a treatment from a group of candidate treatments that corresponds to said individual's CYP2C19 metabolic phenotype; and
c) selecting said treatment.

43. The method of claim 40 wherein said multi-determinant metabolic phenotype is determined according to the method comprising:
a) administering to an individual a probe substrate specific to the CYP2C19 metabolic pathway;
b) detecting metabolites of said CYP2C19 metabolic pathway in a biological sample from said individual in response to said probe substrate; and;
c) characterizing a phenotypic determinant of said CYP2C19 metabolic phenotype based on detected metabolites.

44. A method of screening a plurality of individuals for participation in a drug treatment trial assessing the therapeutic effect of a candidate drug treatment, said method comprising:
a) characterizing a CYP2C19 metabolic phenotype of each of said plurality of individuals;
b) identifying those individuals having a CYP2C19 metabolic phenotype characterized as effective for metabolizing said candidate drug treatment.

45. The method of claim 44 wherein said multi-determinant metabolic phenotype is determined according to the method comprising:
a) administering to an individual a probe substrate specific to the CYP2C19 metabolic pathway;
b) detecting metabolites of said CYP2C19 metabolic pathway in a biological sample from said individual in response to said probe substrate; and;
c) characterizing a phenotypic determinant of said CYP2C19 metabolic phenotype based on detected metabolites.

46. An assay system for detecting the presence of CYP2C19-specific metabolites in a biological sample obtained from an individual treated with a probe substrate specific for CYP2C19 metabolic pathway of said metabolites; said system comprising:
a) means for receiving said biological sample, including an affinity complexation agents contained therein;
b) means for detecting presence of said metabolites bound to said affinity complexation agents; and
c) means for quantifying ratios of said metabolites to provide corresponding phenotypic determinants;
wherein said phenotypic determinants provide a CYP2C19 metabolic phenotype profile of said individual.

47. The assay system of claim 46, wherein said probe substrate is other than an inducer or inhibitor of said metabolic pathway

48. The assay system of claim 47 comprising:
a) means for receiving said biological sample, including an affinity complexation agents contained therein;
b) means for detecting presence of said metabolites bound to said affinity complexation agents; and
c) means for quantifying ratios of said metabolites to provide corresponding phenotypic determinants;
wherein said phenotypic determinants provide a metabolic phenotype profile of said individual and said step b) and step c) are effected according to the method of claim 6.

49. The assay system of claim 48 wherein said means for receiving said biological sample is a multi-well microplate including said affinity complexation agents in each well.

50. The assay system of claim 49 wherein said affinity complexation agents are bound to each well in an array-based format.

51. The assay system of claim 50 wherein said means for detecting said presence of said metabolites bound to said binding agents is a charge-coupled device (CCD) imager.

52. The assay system of claim 51 wherein said means for said quantifying ratios of said metabolites is a densitometer.

53. A method of using a CYP2C19 metabolic phenotype of claim 1 for determining a combination drug therapy wherein an individual's phenotype is indicative of a fast metabolizer, and a corresponding inhibitor is selected for combined treatment with a drug to improve the therapeutic effect thereof in said individual.

54. A method of using a CYP2C19 metabolic phenotype of claim 46 for determining a combination drug therapy wherein an individual's phenotype is indicative of a fast metabolizer, and a corresponding inhibitor is selected for combined treatment with a drug to improve the therapeutic effect thereof in said individual.

55. A method of diagnosing a disease or condition associated with altered function in a drug metabolizing enzyme by determining an individual's CYP2C19 metabolic phenotype.

56. The method of claim 55 wherein said multi-determinant metabolic phenotype is determined according to the method comprising:
a) administering to an individual a probe substrate specific to the CYP2C19 metabolic pathway;
b) detecting metabolites of said CYP2C19 metabolic pathway in a biological sample from said individual in response to said probe substrate; and;
c) characterizing a phenotypic determinant of said CYP2C19 metabolic phenotype based on detected metabolites.

57. A method of determining an individual's susceptibility to a carcinogen induced disease by determining an individual's CYP2C19 metabolic phenotype.

58. The method of claim 57 wherein said multi-determinant metabolic phenotype is determined according to the method comprising:
a) administering to an individual a probe substrate specific to the CYP2C19 metabolic pathway;
b) detecting metabolites of said CYP2C19 metabolic pathway in a biological sample from said individual in response to said probe substrate; and;
c) characterizing a phenotypic determinant of said CYP2C19 metabolic phenotype based on detected metabolites.

59. The method of claim 58 wherein said carcinogen-induced disease is cancer.

60. A method of determining the ability of a compound to effect the function of the CYP2C19 metabolizing enzyme(s) in a biological organism in vivo, said method comprising:
a) determining a first CYP2C19 metabolic phenotype of said biological organism according to the methods of claim 1 prior to exposure to said compound;
b) exposing said biological organism to said compound;
c) determining a second CYP2C19 metabolic phenotype of said biological organism according to the methods of claim 1 after exposure to said compound; and
d) comparing said first and second CYP2C19 phenotypes, wherein a change in said multi-determinant phenotypes determined post-compound exposure as compared to pre-compound exposure is indicative of said drug having the ability to effect the function of said CYP2C19 drug metabolizing enzyme(s) in said biological organism.

61. The method of claim 60, wherein said biological organism is a mammal.

62. The method of claim 61, wherein said mammal is a human.

63. A mephenytoin derivative as is illustrated in FIG. 2.

64. A mephenytoin derivative as is illustrated in FIG. 3.

65. The mephenytoin derivative of claim 63 for use in raising antibodies having an affinity for a chiral form of mephenytoin.

66. The mephenytoin derivative of claim 64 for use in raising antibodies having an affinity for a chiral form of mephenytoin.

67. The mephenytoin derivative of claim 65 for use in detecting a chiral form of mephenytoin in a biological sample.

68. The mephenytoin derivative of claim 66 for use in detecting a chiral form of mephenytoin in a biological sample.

69. The method of claim 1, wherein said step b) or step c) is effected using an affinity complexation agent specific to each of said metabolites.

70. The method of claim 2, wherein said step b) and step c) is effected using an affinity complexation agent specific to each of said metabolites.

71. The method of claim 2, wherein said step b) or step c) is effected using an affinity complexation agent specific to each of said metabolites.

72. The assay system of claim 48 comprising:
a) means for receiving said biological sample, including an affinity complexation agents contained therein;
b) means for detecting presence of said metabolites bound to said affinity complexation agents; and
c) means for quantifying ratios of said metabolites to provide corresponding phenotypic determinants;
wherein said phenotypic determinants provide a metabolic phenotype profile of said individual and said step b) or step c) are effected according to the method of claim 6.

73. The assay system of claim 48 wherein said means for receiving said biological sample is a multi-well microplate including said affinity complexation agents in each well.

74. The assay system of claim 73 wherein said affinity complexation agents are bound to each well in an array-based format.

75. The assay system of claim 74 wherein said means for detecting said presence of said metabolites bound to said binding agents is a charge-coupled device (CCD) imager.

76. The assay system of claim 75 wherein said means for said quantifying ratios of said metabolites is a densitometer.

77. The method of claim 31, wherein said S-mephenytoin is illustrated in FIG. 2.

78. The method of claim 31, wherein said R-mephenytoin is illustrated in FIG. 3.
